(19) United States
(12) Patent Application Publication
(10) Pub. No.: US 2023/0157950 A1
(43) Pub. Date: May 25, 2023

(54) OROMUCOSAL FILM COMPOSITIONS COMPRISING EPINEPHRINE PARTICLES

(71) Applicants: Josef Masek, Brno (CZ); Eliska Masková, Brno (CZ); Robert C H Chisholm, London (GB)

(72) Inventors: Josef Masek, Brno (CZ); Eliska Masková, Brno (CZ); Robert C H Chisholm, London (GB)

(73) Assignees: ReForm Therapeutics Ltd., London (UK); Veterinary Research Institute, Brno (CZ)

(21) Appl. No.: 18/159,320

(22) Filed: Jan. 25, 2023

Related U.S. Application Data
(63) Continuation of application No. 17/934,566, filed on Sep. 22, 2022.
Application No. 17/934,566 is a continuation-in-part of application No. PCT/IB2022/052411, filed on Mar. 17, 2022.
(60) Provisional application No. 63/161,976, filed on Mar. 16, 2021.

(51) Int. Cl. A61K 9/00; A61K 31/167; A61K 31/137; A61K 47/10; A61K 31/445; A61K 9/70; A61K 47/38; A61K 31/381

(52) U.S. Cl. CPC A61K 9/006; A61K 31/167; A61K 31/137; A61K 47/10; A61K 31/445; A61K 9/7007; A61K 47/38; A61K 31/381

(57) ABSTRACT

The invention provides an oromucosal film composition comprising:
a therapeutically effective amount of epinephrine solid particles having a particle size in the range of from about 0.01 um to about 100 um; and
a pharmaceutically acceptable polymer

wherein the epinephrine solid particles are dispersed in and/or disposed on a polymeric layer. The film compositions can be used for the treatment of anaphylactic shock, cardiac arrest, asthma, bronchial asthma, bronchitis, emphysema, respiratory infections, and allergic reactions. The film compositions may further contain local anesthetics and used for providing local anesthesia, such as for the treatment or prevention of tooth pain and treatment of mouth ulcers.

### CROSS REFERENCE TO RELATED PATENT APPLICATION

This application is a continuation of U.S. application Ser. No. 17/934,566 filed Sep. 22, 2022, which is a continuation in part of International Application No. PCT/IB2022/052411 filed Mar. 17, 2022, which claims priority to provisional U.S. Application No. PCT filed Mar. 16, 2021, the contents of which are herein incorporated by reference in their entirety.

### FIELD OF THE INVENTION

This invention relates to oromucosal film compositions comprising epinephrine.

### BACKGROUND TO THE INVENTION

Type 1 hypersensitivity is an allergic reaction caused by improper IgE (Immunoglobulin E) production and activity against typically nonpathogenic antigens, or “allergens”. Anaphylaxis is “a serious allergic reaction that is rapid in onset and may cause death” (Sampson H A. Anaphylaxis: persistent enigma. Emerg Med Australas. 2006 April; 18(2):101-2. doi: 10.1111/j.1742-6723.2006.00816.x), with systemic reactions causing acute onset (minutes to hours) of signs and symptoms in at least 2 organ systems; these include cutaneous (generalized hives, angioedema, flushing, or pruritus), respiratory (throat tightness, dysphagia, hoarseness, change in voice, wheezing, cough, or difficulty breathing), circulatory (dizziness, hypotension, loss of consciousness, or shock), or gastrointestinal (abdominal pain, nausea, or vomiting) symptoms, and can include uterine cramping. Less frequently, cardiac anaphylaxis may cause vasospasm, tachyarrhythmia, or bradycardia (Järvinen K M, Celestin J. Anaphylaxis avoidance and management: educating patients and their caregivers. J Asthma Allergy. 2014 Jul. 10; 7:95-104. doi: 10.2147/JAA.S48611).

Contact with anaphylaxis-inducing agents, and the severity of the resulting anaphylactic reaction, can be extremely unpredictable. Accordingly, allergists recommend that persons who have a personal or family history of anaphylaxis, or a risk of anaphylaxis, be prepared to self-administer emergency treatment at all times. Additionally, adults charged with caring for children who are at risk for anaphylaxis should also be prepared to administer anti-anaphylactic first aid.

Epinephrine products have been approved for use in the United States since 1948, and for use in the emergency treatment of allergic reactions (Type 1) including anaphylaxis since 1987 (EpiPen™, NDA 019430; Mylan Specialty LP). There are currently 4 epinephrine products marketed for the emergency treatment of anaphylaxis: EpiPen and EpiPen Jr™ (NDA 019430), Adrenaclick™ (NDA 020800), Auvi-Q™ (NDA 201739), and Symjepi™ (NDA 207534), all of which are injectable products intended for caregiver or self-administration.

Epinephrine is a commonly administered vasopressor in cardiac arrests (Ayes et al. Epinephrine for Out-of-Hospital Cardiac Arrest: An Updated Systematic Review and Meta-Analysis. Crit Care Med. 2020 February; 48(2):225-229. doi: 10.1097/CCM.0000000000004130). Epinephrine is also a vasoconstrictor that is usually added to extend the duration of local anesthetics, such as those used for dental anaesthesia (Sisk A L. Vasoconstrictors in local anesthesia for dentistry. Anesth Prog. 1992; 39(6):187-93; Moore P A, Hersh E V. Local anesthetics: pharmacology and toxicity. Dent Clin North Am. 2010 October; 54(4):587-99. doi: 10.1016/j.cden.2010.06.015; Becker D E, Reed K L. Local anesthetics: review of pharmacological considerations. Anesth Prog. 2012 Summer; 59(2):90-101; quiz 102-3. doi: 10.2344/0003-3006-59.2.90). Epinephrine has also been used in the treatment of asthma, bronchial asthma, bronchitis, emphysema, respiratory infections, or other allergic reactions.

### Oromucosal Administration

Mucosal drug delivery is an alternative method of systemic drug delivery that offers numerous benefits over parenteral and conventional oral administration. Oral mucosal surfaces have been widely explored for systemic delivery of drugs. Drugs that are absorbed via mucosal surfaces directly enter the systemic circulation and bypass the gastrointestinal tract including first-pass metabolism in the liver. Rapid onset of drug action is an important advantage of mucosal route of administration.

Transmucosal delivery has been employed for administration of therapeutic small molecules and biomacromolecules like peptides, proteins, nucleic acids, as well as antigens and allergens. Transmucosal drug delivery brings many advantages. Oral mucosae, especially its sublingual region, buccal region, and non-keratinized oral regions are attractive sites for non-invasive administration of drugs due to high permeability, a lack of enzymatic barriers, mild pH environment, easy access for self-administration, and opportunities to avoid first-pass metabolism.

Anatomy, physiology, and barrier functions of mucosal surfaces play a critical role in mucosal drug delivery. All aspects should be taken into consideration when designing a mucosal drug delivery system. The mucosa of oral cavity is divided into the buccal, sublingual, gingival, palatal and labial regions. The mucosa of each region is of specific anatomical and functional characteristics. Oral mucosa consists of three layers: a stratified squamous epithelium, composed of several cell layers, below which lies the basement membrane, and finally the connective tissue divided into the lamina propria and submucosa, which comprise numerous vascular capillaries. Drugs absorbed via the oromucosal route of administration are absorbed through these capillaries and gain access to the systemic circulation.

Three major types of epithelium located in different regions of the oral cavity differ in the degree of keratinization—namely masticatory, specialized, and lining mucosa. The masticatory epithelium is keratinized (100-200 μm thick) and covers the gingival region and the hard palate. The specialized epithelium is stratified, keratinized, and covers the dorsal surface of the tongue. The lining mucosa covers buccal and sublingual regions of the oral cavity. The epithelial layer of the buccal and sublingual mucosa is non-keratinized, with variation in thickness 500-600 μm for buccal, 100-200 μm for sublingual mucosa (Hua S. Advances in Nanoparticulate Drug Delivery Approaches for Sublingual and Buccal Administration. Front Pharmacol. 2019 Nov. 5; 10:1328. doi: 10.3389/fphar.2019.01328). The lining mucosa exhibits high permeability for different drugs, and thus is an interesting site for drug administration. The permeability of buccal mucosa is approximately 4-4,000 times greater than that of the skin, but less than that of the intestine.

The oral epithelium is covered by a 70-100 μm thick film of saliva, the secretion from salivary glands. The daily production of saliva secreted into the oral cavity is between 0.5 and 2 mL. Continuous production of saliva significantly impacts drug residence time after administration within the oral cavity, phenomenon known as saliva washout (Patel V F, Liu F, Brown M B. Advances in oral transmucosal drug delivery. J Control Release. 2011 Jul. 30; 153(2):106-16. doi: 10.1016/j.jconrel2011.01.027; Hillery A M, Park K. Drug Delivery: Fundamentals and Applications. 2nd ed. Boca Raton, USA: CRC Press by Taylor & Francis Group, LLC; 2016, 632).

Mucus is the intercellular ground matrix secreted by the sublingual and salivary glands, which is bound to the apical cell surface and acts as a protective layer for the cells below. It is also a visco-elastic hydrogel consisting of the water insoluble glycoproteins, water, and small quantities of different proteins, enzymes, electrolytes and nucleic acids. The mucus layer carries a negative charge due to a high content of the sialic acid and forms a strongly cohesive gel structure that binds to the epithelial cells. The mucus layer varies in thickness from 40 to 300 μm and it plays a critical role in the function of different mucoadhesive drug delivery systems which work on the principle of mucoadhesion, and thus prolong the dosage form retention time at the site of administration.

The rate of drug absorption following oromucosal administration is influenced by the permeability of the buccal and sublingual mucosa, physical-chemical properties of the delivered drug and other factors, namely the presence and properties of mucus, saliva production, movement of the oral tissues during speaking, food and drink intake etc.

Drug permeability through the oral cavity mucosa represents a major limiting factor in transmucosal drug delivery. Mechanically stressed areas are keratinized and impermeable to water, which makes such areas unfavorable for drug delivery. On the other hand, more permeable non-keratinized buccal and sublingual epithelia make such regions of the oral cavity attractive sites for drug delivery and a great number of active ingredients are currently being explored for transmucosal drug delivery (Mašek et al.: Nanofibers in Mucosal Drug and Vaccine Delivery. 2018, IntechOpen, https://doi.org/10.5772/intechopen.82279).

### Transmucosal Delivery of Epinephrine

The sublingual route of administration is a promising alternative route for epinephrine administration. Sublingual formulations of epinephrine would be easy to carry and self-administer eliminating the fear and anxiety associated with needles used in autoinjectors for young children, as well as readily providing the capability of multiple doses. Feasibility studies in humans and animals have shown that epinephrine can be absorbed sublingually (Gu et al., Is epinephrine administration by sublingual tablet feasible for the first-aid treatment of anaphylaxis? A proof-of-concept study. Biopharm Drug Dispos. 2002; 23: 213-216; Simons et al., First-aid treatment of anaphylaxis to food: focus on epinephrine, J Allergy Clin Immunol. 2004;113:425-438). The recommended dose of epinephrine for the treatment of anaphylaxis is about 0.01 mg/Kg: usually about 0.2 mL to about 0.5 mL of a 1:1000 dilution of epinephrine in a suitable carrier.

Epinephrine formulated into oral films comprising an adrenergic receptor interactor and/or a absorption enhancer is described in U.S. Patent Application Publication Nos. 20170348251A1 and 20070293581A1.

Various absorption enhancers were shown to be important excipient in the formulations intended for the delivery of drugs via oral mucosal surfaces (e.g. Nicolazzo et al. Buccal penetration enhancers—How do they really work?, J Control Release. 2005 Jun. 20; 105(1-2):1-15. doi: 10.1016/j.jconrel.2005.01.024., Sohi et al. Critical evaluation of absorption enhancers for oral mucosal drug delivery, Drug Dev Ind Pharm. 2010 March; 36(3):254-82. doi: 10.1080/03639040903117348). Various absorption enhancers were formulated in oral films containing a number of active ingredients including epinephrine in U.S. Patent Application Publication Nos. 20170348251A1 and 20070293581A1.

Rapidly disintegrating sublingual tablets comprising epinephrine nanoparticles or nanocrystals and microparticles or microcrystals are described in U.S. Patent Application Publication Nos. 20190125698 and 20120322884A1.

WO2019165208 is said to disclose a pharmaceutical active-containing single layered, self-supporting, transmucosal delivery device comprising a polymer film comprising a polymer matrix, wherein the film has a pharmaceutical active composition disposed on a surface of the polymer film. The composition has a pH in a range of about 4 to about 9 and comprises at least one pharmaceutical active ingredient in the form of particles, wherein the particles have an average particle size of about 100 nm to about 5 microns. The composition further includes an anti-crystallization agent and a pH adjusting agent. The anti-crystallization agent may comprise various sugar alcohols and di-alcohols, including, for example one or more of sorbitol, mannitol,

### SUMMARY OF THE INVENTION

The present invention provides oromucosal film compositions comprising epinephrine particles, particularly for the emergency treatment of anaphylaxis or a cardiac event. The invention also provides manufacturing methods for preparation of the film compositions.

In one aspect, the present invent provides an oromucosal film composition comprising:
a therapeutically effective amount of epinephrine solid particles having a particle size in the range of from about 0.01 um to about 100 um; and
a pharmaceutically acceptable polymer
   wherein the epinephrine solid particles are dispersed in and/or disposed on a polymeric layer.

In another aspect, the present invent provides an oromucosal film composition consisting essentially of:
a therapeutically effective amount of epinephrine solid particles having a particle size in the range of from about 0.01 um to about 100 um; and
a pharmaceutically acceptable polymer
   wherein the epinephrine solid particles are dispersed in and/or disposed on a polymeric layer.

In yet another aspect, the present invent provides an oromucosal film composition comprising:
a therapeutically effective amount of epinephrine solid particles having a particle size in the range of from about 0.01 um to about 100 um; and
a pharmaceutically acceptable polymer
   wherein the epinephrine solid particles are dispersed in and/or disposed on a polymeric layer, and wherein the composition excludes an anti-crystallization agent, such as various sugar alcohols and di-alcohols, including, for example one or more of sorbitol, mannitol, xylitol, isomalt, and the like.

In some embodiments, the composition is a bi-layered or tri-layered film further comprising a mucoadhesive layer and/or a backing layer. Either the mucoadhesive layer or the polymeric layer comprising epinephrine particles is the middle layer in case of a tri-layered composition. The film is intended for application to oral mucosa with an orientation that keeps the epinephrine particle-containing layer in direct, close contact with the oral mucosa surface.

In some embodiments, the composition further comprises one or more pharmaceutically acceptable excipients selected from the group consisting of pH modifiers, absorption/permeation enhancers, disintegrants, plasticizers, saliva stimulating agent, taste masking agents, flavoring agents, sweeting agent, coloring agent, antioxidants, stabilizers, anti-foaming and/or defoaming components and mixtures thereof. In some embodiments, one or more of the excipients are exclusively or partly present in the polymeric layer in case of a multi-layered composition. In other embodiments, one or more of the excipients are exclusively or partly present in the mucoadhesive layer of a multi-layered composition.

In another aspect, the present invent provides an oromucosal film composition consisting essentially of:
a therapeutically effective amount of epinephrine solid particles having a particle size in the range of from about 0.01 um to about 100 um;
one or more absorption enhancers;
one or more pH modifiers; and
a pharmaceutically acceptable polymer
   wherein the epinephrine solid particles are dispersed in and/or disposed on a polymeric layer.

In some other embodiments, the composition further comprises one or more local anesthetics in the polymeric layer and/or the mucoadhesive layer.

In another aspect, the invention provides a method for preparation of an oromucosal film composition comprising:
providing a suspension of epinephrine solid particles having a particle size in the range of from about 0.01 um to about 100 um in a solvent;
simultaneously or separately providing a suspension or solution of a pharmaceutically acceptable polymer in a solvent;
combining the epinephrine solid particle suspension and the polymer suspension/solution if prepared separately; and

removing the solvent(s) to obtain the polymeric layer.

In yet another aspect, the oromucosal film compositions of the invention may be prepared by preparation methods comprising solvent casting, hot melt extrusion, electro spinning, 3-dimensional or flexographic printing, spraying, combination of solvent casting and spraying, electrospraying, blow spinning, electroblowing, centrifugal spinning, or a combination of electrospinning and electrospraying.

In another aspect, a method of treating anaphylactic shock in a subject in need thereof is provided comprising administering a film composition of the invention to the subject.

In yet another aspect, a method of treating cardiac arrest, asthma, bronchial asthma, bronchitis, emphysema, respiratory infections, or allergic reactions in a subject in need thereof is provided comprising administering a film composition of the invention to the subject.

In another aspect, a method of providing local anesthesia in a subject in need thereof comprising administering to the subject a film composition according to the invention.

In yet another aspect, a method of treating and/or preventing tooth pain and/or treating mouth ulcers in a subject in need thereof is provided comprising administering a film composition of the invention comprising epinephrine particles and one or more local anesthetics to the subject. A lower dose of epinephrine may be used in combination with local anesthetics for such purposes in conjunction with a slower dissolving polymer to prolong anesthesia. Local anesthetics may include lodicaine, novocaine, articaine, prilocaine, mepivacaine, salts and mixtures thereof,

In an alternative aspect of the invention, when a low dose of epinephrine is used in combination with local anesthetics, the epinephrine need not be in solid particulate form and an absorption enhancer selected from the group consisting of diethylene glycol monoethyl ether, eugenol, bile acids, caprylocaproyl polyoxyl-8 glycerides, disodium ethylenediaminetetraacetic acid salt, and mixtures thereof, may be used to in preparing the oromucosal film.

### BRIEF DESCRIPTION OF THE DRAWINGS

FIG. 1 depicts alternative types of epinephrine particle-containing oromucosal films of the invention.

FIG. 2 depicts structure of epinephrine-containing polymeric layer of an oromucosal film: A shows a conventional film; B shows four alternative films of the invention; C depicts top view and cross-sectional views of films of the invention having polymeric fiber layer(s).

FIG. 3 depicts three alternative types of epinephrine particle-containing oromucosal films of the invention comprising pharmaceutical excipients film.

FIG. 4 shows the size distribution of the epinephrine base particles: A—source/original (Cambrex) particles; B—particles after 1 cycle of micronization in the microfluidizer; C—particles after 4 cycles of micronization in the microfluidizer.

FIG. 5 shows the structure and size of the original epinephrine base particles (source raw material) before any processing involving solvents/dispersants observed using scanning electron microscopy (SEM).

FIG. 6 shows the impact of processing with selected dispersants on epinephrine base particles at specified temperatures for specified durations observed using SEM.

FIG. 7 shows the impact of epinephrine concentration on the structure and size of epinephrine base particles when processed with dispersants observed using SEM.

FIG. 8 shows the effect of processing using water having neutral or acidic pH on the structure and size of epinephrine base particles at a specified temperature for specified durations observed using SEM.

FIG. 9 shows epinephrine base particle-containing polymeric films of the invention observed using SEM: A—film manufactured using a solution of citric acid in acetone as a solvent/dispersant; B—enlarged details of A; C—film manufactured using ethanol with sodium deoxycholate as a solvent/dispersant; D—enlarged detail of C.

FIG. 10 shows the effect of using absorption enhancers and pH modifiers in the oromucosal film compositions of the invention on the dissolution rate and solubility of epinephrine base particles in the dissolution medium.

FIG. 11 shows the percentage amount of dissolved and released epinephrine from different oromucosal films of the invention where the amount released after 1 hour was considered as 100%.

FIG. 12 shows the effect of absorption enhancers and pH modifiers on the absorption rate of epinephrine directly released from an oromucosal film of the invention comprising epinephrine base particles using a diffusion cell.

FIG. 13 shows epinephrine concentration in blood plasma of piglets following administration of a control/placebo film.

FIG. 14 shows concentration of epinephrine in blood plasma of piglets following sublingual administration of the oromucosal film composition of the invention containing 8 mg of epinephrine base particles.

FIG. 15 shows concentration of epinephrine in blood plasma of piglets following EpiPen 0.3 mg administration.

FIG. 16 shows the level of epinephrine in the blood plasma of a rabbit following sublingual administration of oromucosal films of the invention formulated with epinephrine base particles and a placebo film composition.

FIG. 17 shows the design of a bi-layered mucoadhesive orodispersible film including a thin binding layer formed using a binding solution.

FIG. 18 shows NMR analysis of the crystalline state of Epinephrine base in the mucoadhesive film formulations.

FIG. 19 shows dissolution of Epinephrine in the mucoadhesive layer following the application of mucoadhesive film with the citric acid-containing backing layer onto oral mucosa ex vivo.

FIG. 20 shows pharmacokinetic profile of Lidocaine following administration of Epinephrine and Lidocaine containing films of the invention.

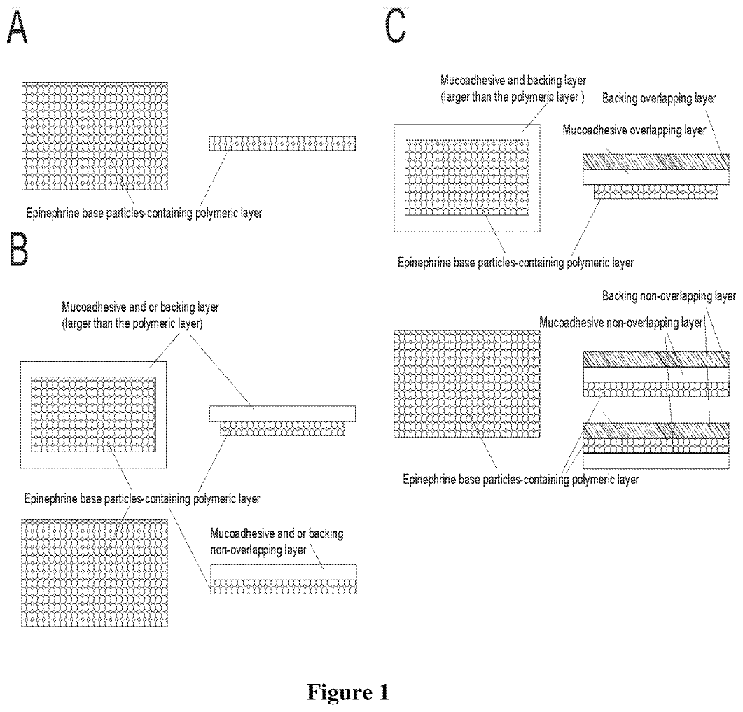

### DETAILED DESCRIPTION OF THE INVENTION

The epinephrine particles used in the invention may be in the form of epinephrine free base, epinephrine acid addition salts, or mixtures thereof. Salts of epinephrine include bitartrate, and hydrochloride acid addition salts. Epinephrine base and salts are well known in the art and may be prepared by convention methods or obtained commercially.

The term “consisting essentially of” means that the composition is limited to the components specified and those additional components that do not materially affect the basic and novel characteristic(s) of the composition.

The epinephrine solid particles are dispersed in and/or disposed on a polymeric layer which is a consistent homogeneous film or porous layer comprising of one or more polymers. In some embodiments, epinephrine particles are embedded within the polymeric layer, thus forming a composite material of polymers and particles. In some other embodiments, epinephrine particles are attached to, or disposed on the polymeric layer.

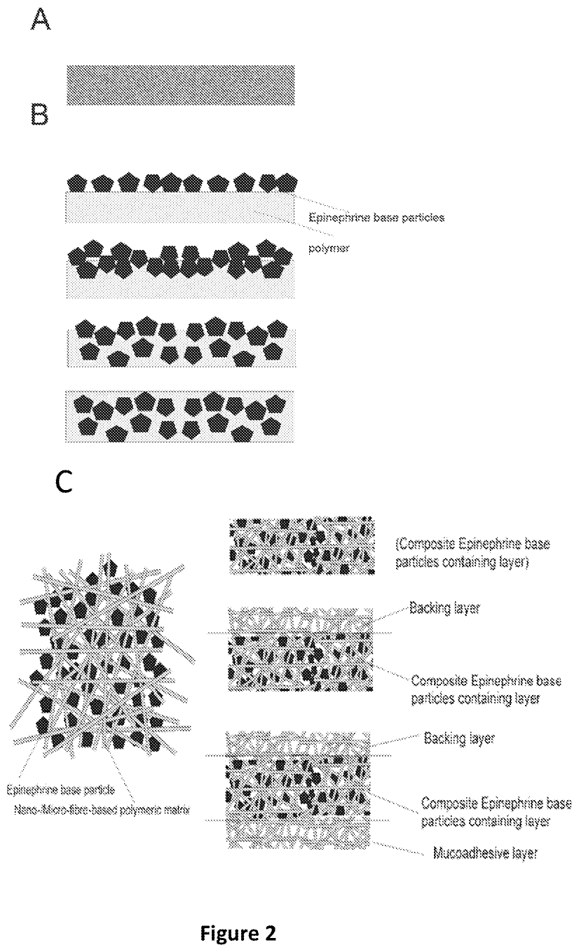

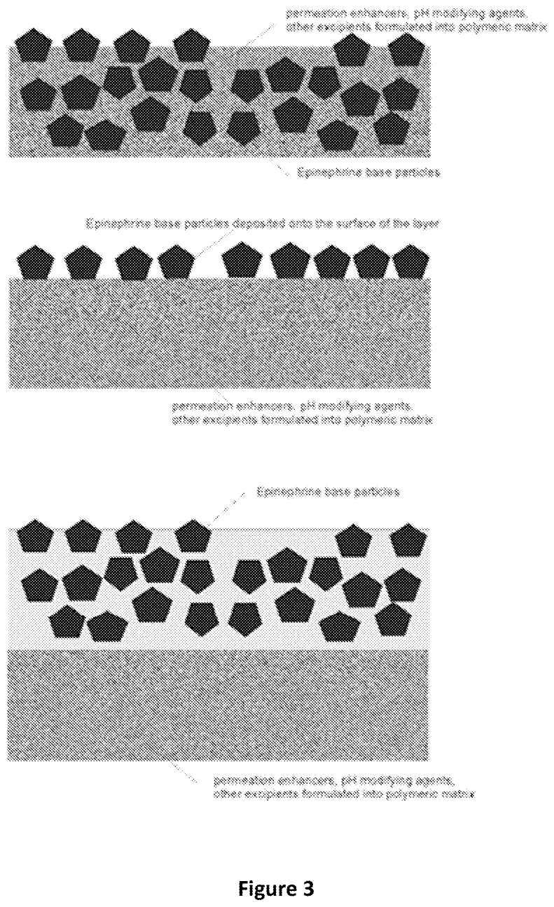

Epinephrine particles may be distributed uniformly throughout the polymeric layer, disposed or embedded on the entire surface of the polymeric layer or only a portion of its surface, as shown in FIGS. 2B, 3 and 9. The distribution of epinephrine base particles may or may not be homogeneous. In contrast, a convention film composition, as shown in FIG. 2A, has epinephrine base or its soluble salts formulated into a polymeric matrix such that epinephrine molecules are homogeneously distributed in the polymeric matrix, such as in a solid dispersion system.

In some embodiments, the polymeric layer is a porous layer comprising polymeric nanofibers and/or microfibers. When nano or microfibers are used, epinephrine particles are deposited on the surface of fibers, and/or deposited into a mesh of polymeric nano or microfibers as shown in FIG. 2C. The composite fiber-based polymeric film can be single-, bi- or tri-layered, where there is a backing layer, composite epinephrine particle-containing layer (with or without mucoadhesive properties) and a layer of mucoadhesive micro or nanofibers. The backing layer can be the same size as the other layers or larger to form a mucoadhesive seal around the other layers.

In some embodiments epinephrine solid particles are at least partly included or embedded within the polymeric microfiber and/or nanofiber. In some other embodiments, epinephrine solid particles are dispersed in the polymeric layer but are not part of or included within the polymeric microfibers and/or nanofibers.

The epinephrine particle-containing polymer layer may be designed to dissolve in about 1 to about 30 minutes in the mouth, including more than 1 minute, more than 5 minutes, more than 10 minutes, more than 20 minutes or less than 30 minutes. In some embodiments, such as when used for local anesthesia, such as dental anesthesia, the polymer layer may dissolve in more than 30 minutes and last up to 12 hours in the mouth.

For fast dissolving films low molecular weight hydrophilic polymers, such as polymers having a molecular weight between about 1,000 to 9,000 daltons, or polymers having a molecular weight up to 900,000 daltons may be used, optionally in combination with other suitable polymers. For slower dissolving films higher molecular weight polymers, such as those having a molecular weight in millions of daltons may be used, optionally in combination with other suitable polymers. Desired results can be achieved by adjusting the concentration of two or more types of polymers. Concentration of the polymers and the type/nature/solubility of the polymers used in each layer of the composition may be varied to control the amount of time the film composition resides on the mucosa. Disintegrants and plasticizers may be used to adjust erodibility of the film composition. Water soluble pharmaceutically acceptable excipients may be used to achieve desired erodibility/solubility of the film, particularly when insoluble or low solubility polymers are included.

In certain embodiments, pH modifiers are formulated into the epinephrine particle-containing polymer layer. In certain other embodiments, pH modifiers are formulated into the mucoadhesive and/or backing layer.

In certain embodiments, absorption enhancers are formulated into the epinephrine particle-containing polymer layer. In certain other embodiments, absorption enhancers are formulated into the mucoadhesive or backing layer.

In certain embodiments, absorption enhancers, pH modifying agents and epinephrine particles are mixed with the polymer to formulate a polymeric matrix so as to form a composite pharmaceutical film formulation. In certain other embodiments, absorption enhancers, pH modifying agents are formulated into a polymeric matrix comprising nano/microfibers and epinephrine particles are deposited in the polymeric matrix. In certain embodiments, absorption enhancers and pH modifying agents are formulated into a polymeric matrix and epinephrine particles are deposited or deposed on the polymeric matrix.

In certain embodiments, when polymeric nano/microfibers are used, mucoadhesive polymers are used in forming the fibers.

The weight ratio of epinephrine to the polymer(s) used in the polymeric layer may be from about 5% to about 50%, from about 10% to about 40%, from about 15% to about 35%, from about 15% to about 30%, or from about 15% to about 25%.

While epinephrine solid particles dispersed in and/or disposed on the polymeric layer have a particle size in the range of from about 0.01 um to about 100 um, in some embodiments the particle size may be from about 0.1 um to about 50 um, from about 1 um to about 40 um, from about 8 um to about 100 um, from about 8 um to about 80 um, from about 10 um to about 100 um, from about 10 um to about 80 um, from about 20 um to about 80 um, from about 20 um to about 30 um, or from about 0.1 um to 3 um.

In some embodiments, the polymers used in the polymeric layer may be rapidly or moderately dissolvable in the saliva. In some embodiments, the polymers may be water soluble or swellable. Suitable polymers include water-soluble synthetic polymers and co-polymers, polosynthetic polymers and co-polymers, and natural polymers. Examples of suitable polymers include cellulose derivatives, polyacrylates, poloxamers, polyethylene oxides, polyvinyl alcohols, povidone, poly-amino acids, gums and other natural polymers.

Polymers that may be used in the polymeric layer include hypromellose, hydroxypropyl cellulose, hydroxyethyl cellulose, carboxymethyl cellulose, carboxymethyl cellulose sodium, croscarmellose sodium, carbomer, polycarbophil, povidone, crospovidone, polyvinyl alcohol, polyethylene oxide, polyethylene glycol, sodium alginate, calcium alginate, xanthan gum, pectin, hyaluronic acid, sodium hyaluronate, tragacanth, guar gum, acacia gum, arabic gum, lectin, starch, gelatin, pullulan, carrageenan, chitosan, amino methacrylate copolymers, poloxamer, collagen, and mixtures thereof.

In case of a single layered or bi-layered film composition comprising a backing layer, the polymers used in the polymeric layer include one or more mucoadhesive polymers. A suitable mucoadhesive polymer is also used in the mucoadhesive layer of a bi-layered or tri-layered film.

Mucoadhesive polymers in the polymeric or mucoadhesive layer are used to enhanced retention of the film composition on the oral mucosa. Such polymers include soluble and insoluble, non-biodegradable and biodegradable polymers. These polymers can be hydrogels or thermoplastics, homopolymers, copolymers or blends, natural or synthetic. Examples of suitable polymers include the same polymers as listed above for polymeric layer, provided they have mucoadhesive properties.

A backing layer is an occlusive layer designed to drive unidirectional release and absorption of epinephrine into the mucosa thereby decreasing amount of active ingredient required. A backing layer can also improve mechanical properties of the film to facilitate handling during production and use.

Backing layer may be formed using a hydrophobic, hydrophilic, erodible, low solubility or insoluble polymer, such as ethyl cellulose, hydroxyethyl cellulose, hydroxypropyl cellulose, methylcellulose, hypromellose, hypromellose phthalate, cellulose acetate, cellulose acetate phthalate, cellulose acetate butyrate, polyvinyl alcohol, povidone, crospovidone, polyethylene glycol, polyethylene oxide, polyvinyl alcohol-polyethylene glycol graft-copolymers, amino methacrylate copolymers, ethylene oxide, propylene oxide co-polymers, poly(vinyl acetate), poly(ethylene-co-vinyl) acetate, polycaprolactone, pharmaceutical wax, and mixtures thereof.

The mucoadhesive layer and/or backing layers may fully overlap with the polymeric layer or be larger than the polymeric layer such that they extend beyond and encompass the polymeric layer on all sides to facilitate effective mucoadhesion and/or prevent peripheral loss of epinephrine.

In some embodiments, the polymeric layer comprising the epinephrine solid particles is printed onto the mucoadhesive and/or backing layer.

One or more local anesthetics may be included in the polymeric layer and/or the mucoadhesive layer when the film composition is intended for providing local anesthesia, such as for treating tooth pain or mouth ulcers. Suitable local anesthetics include lidocaine, articaine, bupivacaine, prilocaine, mepivacaine, and mixtures thereof.

The thickness of the film composition may vary, depending on the number of layers and their thickness. The thickness and number of layers may be adjusted in order to vary the release/absorption of epinephrine particles and erosion/residence of the film in the mouth. For example, the thickness may range from about 0.05 μm to about 2 mm, from about 0.1 to about 0.5 mm, from about 0.01 mm to about 1 mm, or greater than 0.1 mm, greater than 0.2 mm, or less than 0.5 mm, less than 0.2 mm, less than 0.1 mm, or less than 1 um. The thickness of each layer may vary from 0.1 to 99.9% of the overall thickness, or 1 to 99% of the overall thickness, or 10 to 90% of the overall thickness of the multi-layered composition. For example, the thickness of each layer may vary from 0.01 mm to 0.9 mm.

### Pharmaceutically Acceptable Excipients

The film composition may further comprise one or more pharmaceutically acceptable excipients, such as pH modifiers, absorption enhancers, binders, disintegrants, plasticizers, saliva stimulating agent, taste masking agents, flavoring agents, sweeting agent, coloring agent, antioxidants, stabilizers, anti-foaming and/or defoaming components and mixtures thereof. In some embodiments, the excipients are exclusively or partly present in the mucoadhesive layer and/or backing layer in case of a multilayered film. These excipients are well known in the art, such as those described in US20170348251A1.

The excipients may constitute up to about 80%, such as about 0.005% to 50%, or from about 1% to about 30% by weight of the composition. Epinephrine base may comprise

In some embodiments, pH modifiers are used to enhance the bioavailability of epinephrine. include weak organic acids including amino acids, phosphoric acid, acidic polymers, and salts and mixtures thereof.

Weak organic acids include citric acid, acetic acid, succinic acid, tartaric acid, maleic acid, malic acid, ascorbic acid, acetylsalicylic acid, adipic acid, fumaric acid, glutaric acid, glutamic acid, itaconic acid, aspartic acid, lactic acid, and salts and mixtures thereof. Salts of acids include inorganic salts, such as alkali metal salt, alkaline earth metal salt, ammonium salt, and salts formed with an organic base, such as lysine, arginine and meglumine.

In some embodiments, the pH modifiers are acidic polymers which serve the dual role of forming the polymeric layer and pH modification. Suitable acidic polymers include carboxymethylcellulose, cross-linked carboxymethylcellulose, poly(acrylic acid) polymers and copolymers, alginic acid, sodium alginate, calcium alginate, hyaluronic acid, mixtures and salts thereof.

In some embodiments, absorption enhancers are used to increase the bioavailability of epinephrine. Suitable absorption enhancers include chelators, non-ionic surfactants, cationic surfactants, anionic surfactants, bile salts and other steroidal detergents, fatty acids, fatty acids salts and esters, sucrose fatty acid esters, non-surfactants, phospholipids, complexing agents, cyclodextrins, alkyl glycosides, polyethylene glycol alkyl ethers, self-emulsifying agents, and mixtures thereof. Specific absorption enhancers include limonene, menthol, pinene, clove oil, eugenol, caprylocaproyl polyoxyl-8-glycerides, propylene glycol monocaprylate, deoxycholate sodium, taurocholate sodium, glycocholate sodium, diethylene glycol monoethyl ether (Transcutol® HP), dodecylmaltoside, tetradecyl maltoside, Intravail®, and mixtures thereof.

Flavoring agents and/or sweeteners include acacia syrup, acesulfame K, alitame, anise, apple, aspartame, banana, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza syrup, grape, grapefruit, honey, isomalt, lemon, lime, lemon cream, monoammonium glyrrhizinate, maltol, mannitol, maple, marshmallow, menthol, mint cream, mixed berry, neohesperidine DC, neotame, orange, pear, peach, peppermint, peppermint cream, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, swiss cream, tagatose, tangerine, thaumatin, tutti fruitti, vanilla, walnut, watermelon, wild cherry, wintergreen, xylitol, or mixtures thereof.

Antioxidants include tocopherols and the esters thereof, sesamol of sesame oil, coniferyl benzoate of benzoin resin, nordihydroguaietic resin and nordihydroguaiaretic acid, gallates, butylated hydroxyanisole, ascorbic acid and salts and esters thereof, such as acorbyl palmitate, erythorbinic acid and salts and esters thereof, monothioglycerol, sodium formaldehyde sulfoxylate, sodium metabisulfite, sodium bisulfate, sodium sulfite, potassium metabisulfite, butylated hydroxy toluene, propionic acid, and combinations thereof.

Stabilizers include calcium chloride, calcium disodium ethylene diamine tetra-acetate, glucono delta-lactone, sodium gluconate, potassium gluconate, sodium tripolyphosphate, sodium hexametaphosphate, as cyclodextrins, cyclomannins (5 or more α-D-mannopyranose units linked at the 1,4 positions by α linkages), cyclogalactins (5 or more β-D-galactopyranose units linked at the 1,4 positions by β linkages), cycloatrins (5 or more α-D-altropyranose units linked at the 1,4 positions by α linkages), and combinations thereof.

Suitable coloring agents include azo dyes, organic or inorganic pigments, such as the oxides or iron or titanium, or coloring agents of natural origin. Anti-foaming and/or de-foaming agents include simethicone and similar agents.

Plasticizers include polyethylene glycols, polypropylene glycols, polyethylene-propylene glycols, glycerol, glycerol monoacetate, diacetate or triacetate, triacetin, polysorbate, cetyl alcohol, propylene glycol, sorbitol, sodium diethylsulfosuccinate, triethyl citrate, tributyl citrate, phytoextracts, fatty acid esters, fatty acids, and mixtures thereof.

Binders include starches, natural gums, pregelatinized starches, gelatin, polyvinylpyrrolidone, methylcellulose, sodium carboxymethylcellulose, ethyl celluiose, polymerylamides, polyvinyloxoazolidone, polyvinyl alcohols, and mixtures thereof.

The amount of epinephrine base used in the epinephrine base particle-containing layer may be from about 10% to about 30%, from about 12% to about 25%, from about 15% to about 20%, from about 15% to about 30%, or from about 15% to about 25%.

The amount of polymer used in the polymeric layer may be from about 5% to about 90%, from about 10% to about 85%, from about 25% to about 85%, from about 50% to about 75%, from about 55% to about 75%, or from about 60% to about 70%. The amount of absorption enhancer used in the polymeric layer may be from about 1% to about 3%, from about 1% to about 20%, from about 2% to about 15%, from about 6% to about 12%, or from about 3% to about 10%. The amount of antioxidant used in the polymeric layer may be from about 0.001% to about 10%, from about 0.005% to about 5%, from about 0.01% to about 3.5%, or from about 0.1% to about 3%. The amount of plasticizer used in the polymeric layer may be from about 2% to about 20%, from about 3% to about 18%, from about 4% to about 17%, from about 5% to about 20%, or from about 12% to about 18%.The amount of pH modifier used in the polymeric layer may be from about 1% to about 25%, from about 2% to about 20%, from about 3% to about 18%, from about 4% to about 17%, or from about 5% to about 15%.

### Preparation Methods

The film compositions of invention may be prepared by conventional methods, such as solvent casting, hot melt extrusion, spinning, electro spinning, 3-dimensional or flexographic printing, spraying, electrospraying, combination of solvent casting and spraying, a combination of electrospinning and electrospraying, or any other permutation or combination of these methods. For example, methods described in Boda et al., J Aerosol Sci. 2018 November; 125: 164-181; Nguyen et al., J. Parm. Sci. 2016; 105: 2601-2620; Soares et al., Mater. Sci. Eng. C 2018 November; 92(1): 969-982; and Janßen et al., Int. J. Pharm. 2013 January; 441(1-2): 818-825 may be used.

In some embodiments, micronized particles of epinephrine are used.

The film may be formulated as a dispersion of epinephrine solid particles in a polymeric solution, such as by solvent casting method, or epinephrine particles are deposited into/onto the polymer layer by alternative methods, such as electrospinning, electrospraying, hot-melt extrusion, printing techniques (e.g., flexographic printing) or spraying, combination of solvent casting and spraying, solution blow spinning, electroblowing, centrifugal spinning, a combination of electrospinning and electrospraying, or any other suitable permutation or combination thereof.

Polymeric micro/nanofibers are produced by electrospinning, or other alternative methods (e.g. solution blowing, spinning method). Epinephrine particles are deposited using alternative spraying methods, such as electrospraying, simultaneously, or consecutively when producing polymeric fibers.

In case of a multi-layered film compositions, the layers may be manufactured separately and then laminated or layered in succession. Binding solution of polymer(s) may be sprayed between two film layers, such as the polymeric and backing layer to combine the layers. The binding solution can contain excipients, such as mucoadhesive polymer, pH modifier, and/or absorption enhancer, thereby forming the third layer of a three-layered system in situ.

In some embodiments, in case of a multi-layered film, the polymeric layer comprising the epinephrine solid particles is printed onto the mucoadhesive and/or backing layer, such as by employing a 3-dimensional or flexographic printing method. The mucoadhesive and/or backing layers may also be prepared by 3-dimensional or flexographic printing.

In some embodiments, the preparation method comprises:
providing a suspension of epinephrine solid particles having a particle size in the range of from about 0.01 um to about 100 um in a solvent/dispersant;
simultaneously or separately providing a suspension or solution of the polymer in a solvent/dispersant;
combining the epinephrine solid particle suspension and the polymer suspension/solution if prepared separately; and
removing the solvent/dispersant to obtain the polymeric layer.

Solvents/dispersants are used in the manufacturing process of oromucosal films of the invention to dissolve polymers and pharmaceutical excipients including plasticizers, pH modifiers, stabilizers, antioxidants, taste masking agents and absorption enhancers; all or just some of these excipients may be used depending on the requirements of the film formulation. In some embodiments, one or more polymers may remain suspended/dispersed or partially dissolved. In some embodiments, one or more excipients may remain suspended/dispersed or partially dissolved.

During the manufacturing process, those solvents/dispersants are used when processing epinephrine that do not dissolve epinephrine particles to any appreciable extent. The solvents/dispersant should disperse solid particles of epinephrine base without substantial dissolution to ensure the vast majority of epinephrine base is maintained as particulate in nature without any recrystallization, degradation or oxidization. More specifically, epinephrine particles are insoluble, practically insoluble, very slightly soluble or slightly soluble (as defined in the United States Pharmacopeia) in the solvents/dispersant used.

Suitable solvent/dispersant used in the preparation methods of the invention include lower alkyl alcohols, acetone, methyl acetate, ethyl acetate, isopropyl acetate, acetonitrile, heptane, tetrahydrofuran, water, alkaline water, aqueous buffer, and mixtures thereof. Suitable lower alkyl alcohols include methanol, ethanol, isopropyl alcohol, 2-propanol, 1-propanol 1-butanol, t-butanol, and mixtures thereof.

In some embodiments an aqueous dispersion of epinephrine base solid particles is prepared, and the pH of the dispersion is maintained above about 8 using a base or an alkali to prevent dissolution of the particles. For example, the pH of the dispersion may be maintained between about 8.5 to about 9.5.

### Use

The film compositions of the invention may be used for the treatment of anaphylactic shock, cardiac arrest, asthma, bronchial asthma, bronchitis, emphysema, respiratory infections, and allergic reactions. The composition may be administered sublingually, buccally or via any other suitable mucosal surface.

When a local anesthetic is included, the film compositions of the invention may be used for the providing for local anesthesia, such as for treatment and/or prevention of tooth pain and treatment of mouth ulcers. A lower dose of epinephrine is used in combination with local anesthetics, such as lodicaine, novocaine, articaine, prilocaine, or mepivacaine, in a slower dissolving polymer for local or dental anesthesia. The amount of epinephrine base used in epinephrine base particle-containing layers in such cases may be from about 0.05% to about 5%, from about 0.1% to about 2%, from about 0.25% to about 1%, or from about 0.5% to about 1%. The amount of local anesthetic used in epinephrine base particle-containing layers in such cases may be from about 5% to about 90%, from about 10% to about 85%, or from about 50% to about 80%, or from about 50% to about 60%. The amount of polymer used in the polymeric layer may be from about 5% to about 40%, from about 10% to about 35%, or from about 15% to about 30. The amount of plasticizer used in the polymeric layer may be from about 1% to about 10%, from about 2% to about 8%, from about 2% to about 5%, or from about 2% to about 3. In addition to providing drug delivery, once the film composition adheres to the mucosal surface, it may also provide protection to the treatment site by acting as an erodible bandage.

When a low dose of epinephrine is used in combination with local anesthetics, and an absorption enhancer selected from the group consisting of diethylene glycol monoethyl ether, eugenol, bile acids, caprylocaproyl polyoxyl-8 glycerides, disodium ethylenediaminetetraacetic acid salt, and mixtures thereof, the epinephrine need not be in solid particulate form for preparing the oromucosal film. The quantity of ingredients used and the manner and design of film composition may be the same as that describes herein when solid particles of epinephrine are used. Epinephrine and one or more of local anesthetics may be present in a single layer or in separate layers. Epinephrine and one or more of local anesthetics may be dispersed or dissolved in and/or disposed on a polymeric layer which may be mucoadhesive.

### EXAMPLES

The examples demonstrate that the epinephrine base particles maintain their structure when formulated into film using methods of the invention and that epinephrine base particles are compatible with absorption enhancers, and other excipients when formulated into film compositions of the invention. The examples also show that epinephrine is released rapidly from the film compositions of the invention and is quickly and effectively absorbed, as demonstrated under both ex-vivo and in-vivo conditions.

### Example 1

### The Effect of Different Solvents/Dispersants on the Size and Structure of Epinephrine Base Particles

### Size Distribution of Epinephrine Particles

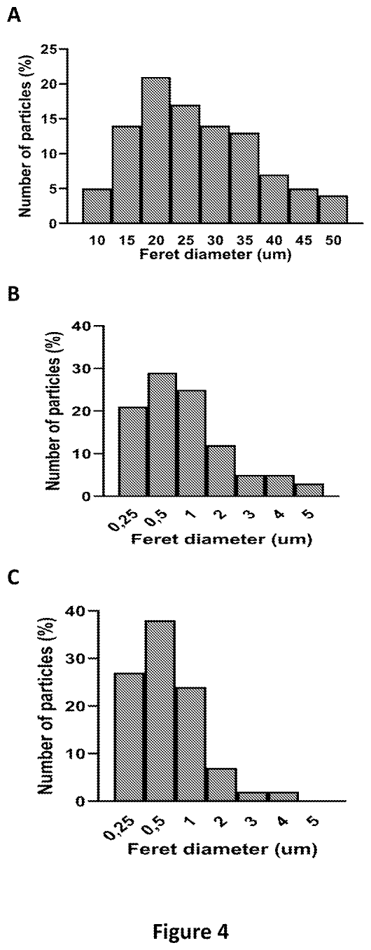

The epinephrine base particles were obtained as active pharmaceutical ingredient from Cambrex. The particles were micronized through microfluidization. The original and modified particles were evaluated for particle size distribution prior to formulation into film compositions. The results of are depicted in FIG. 4. The as supplied epinephrine base particles (Cambrex) having particle size distribution depicted in FIG. 4A were used in the preparation and testing/evaluation of all films described in the examples below unless otherwise specified.

### Methods

### Size Distribution Determination

The particle size distribution of starting/original epinephrine base particles (as supplied by Cambrex), and those obtained after micronization were analyzed using pictures taken by scanning electron microscopy (Hitachi SU8020, Japan). The size of particles was expressed as a Feret's diameter measured in a fixed direction.

### Micronization

Epinephrine base particles (Cambrex) were subjected to micronization using Microfluidizer LV-1 (Microfluidics) for one or four cycles. Briefly, epinephrine base particles were suspended in alkaline water or isopropanol (50 mg/ml) and the pressure was set to 1200 PSI. One or four cycles of microfluidization were performed. Sodium metabisulphite was added into the suspension to prevent epinephrine oxidation during the process.

### SEM Observation

The particles were observed using scanning electron microscopy. The samples were prepared by deposition of the sample on a glass surface, evaporation of solvent in the sample, and sputter coating using platinum/palladium electrode.

### Results

FIG. 4A shows the size distribution of the source/original epinephrine base particles (Cambrex). FIG. 4B shows the size distribution of epinephrine base particles after 1 cycle of micronization in the microfluidizer. 4C shows the size distribution of epinephrine base particles after 4 cycles of micronization in the microfluidizer.

### Conclusion

The original particle size was successfully reduced from about the size of 20 um to about 2 um or less using one or more cycles of microfluidization.

### Solubility of Epinephrine Particles

Solubility experiments were performed to confirm the suitability of solvents/dispersants for manufacturing the film compositions of the invention. Solid particles of epinephrine base were subjected to solubility tests at increasing time intervals and temperatures using selected solvents/dispersants suitable for use in industrial pharmaceutical manufacturing processes. Selected solvents/dispersants dissolve film-forming excipients, plasticizers and eventually other pharmaceutical excipients including pH modifiers, stabilizers, antioxidants, taste masking agents, absorption enhancers but disperse epinephrine particles. The solubility of epinephrine particles may not be higher than 10% weight/volumes in the solvent/dispersant used. Epinephrine particles are very slightly soluble in some of the selected solvents/dispersants and preferably practically insoluble in some of the other selected solvents/dispersants.

Epinephrine solubility experiments were performed under various experimental conditions corresponding to those occurring during the manufacture of conventional oromucosal films. The effect of time, temperature and the concentration of epinephrine were evaluated using selected solvents/dispersants. Solvents/dispersants were selected from acetone, ethanol, ethyl acetate, 1-butanol, t-butanol, isopropyl alcohol, isopropyl acetate, methanol, 2-propanol, 1-propanol or acetonitrile, heptane, tetrahydrofuran, alkaline water-based solvents (water, buffers of pH 7, 8, 8.5, 9) and mixtures with water-miscible solvents thereof.

### Method

Acetone, ethanol, methanol, isopropyl alcohol and alkaline water-based solvents of pH 7, 8, 8.5 and 9 were chosen as suitable solvents/dispersant candidates for the screening. Their effect on the solubility of epinephrine base particles was observed using scanning electron microscopy. Citric acid solution and water solution acidified using hydrochloric acid (HCl) of pH 2 were used as examples of standards/comparative solvents that dissolve epinephrine particles. The dissolution was followed by crystallization by evaporating the solvent prior to scanning with SEM.

To confirm the level of epinephrine base solubility at different concentrations, suspensions of epinephrine base particles at concentrations of 0.5 g/L, 5 g/L and 50 g/L were prepared in solvent/dispersants and the mixture gently stirred at 25° C. and 45° C. After this step, the solvent/dispersant was evaporated at 25° C. or 45° C. and the size and structure of the epinephrine particles obtained was evaluated using scanning electron microscopy (SEM, HITACHI SU 8020, Japan). Samples of the mixtures were withdrawn at 60, 180 and 360 minutes and the structure of the epinephrine particles was evaluated.

### Results

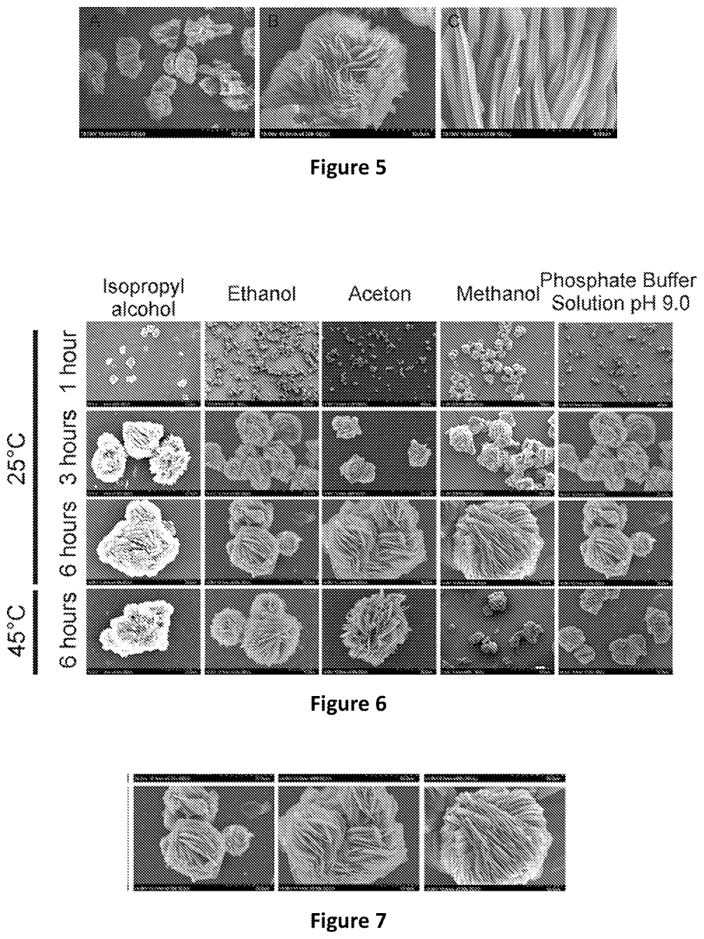

FIG. 5 shows the structure and the size of the original particles (source raw material) before being exposed to any solvents/dispersants. FIG. 6 examines the effect of the selected dispersants on Epinephrine base particles. The selected dispersants had no impact on the nanostructure of the epinephrine base microparticles. The nanostructured morphology of the epinephrine particles composed of sheets of the thickness of tens to hundreds of nanometers is clearly visible before (FIG. 5) and after the dispersion/processing of the particles (FIG. 6) using the selected solvents/dispersants. The electron micrographs clearly show that epinephrine particles kept their original structure confirming none or very limited solubility at 25° C. and 45° C. for a duration of up to 6 hours of exposure to the solvents/dispersant.

FIG. 7 examines the effect of the three different epinephrine concentration on the structure and the size of the Epinephrine particles. The results confirmed that the concentration of epinephrine base particles in the dispersant/solvents had no influence on the physical size and structure of the processed epinephrine base particles.

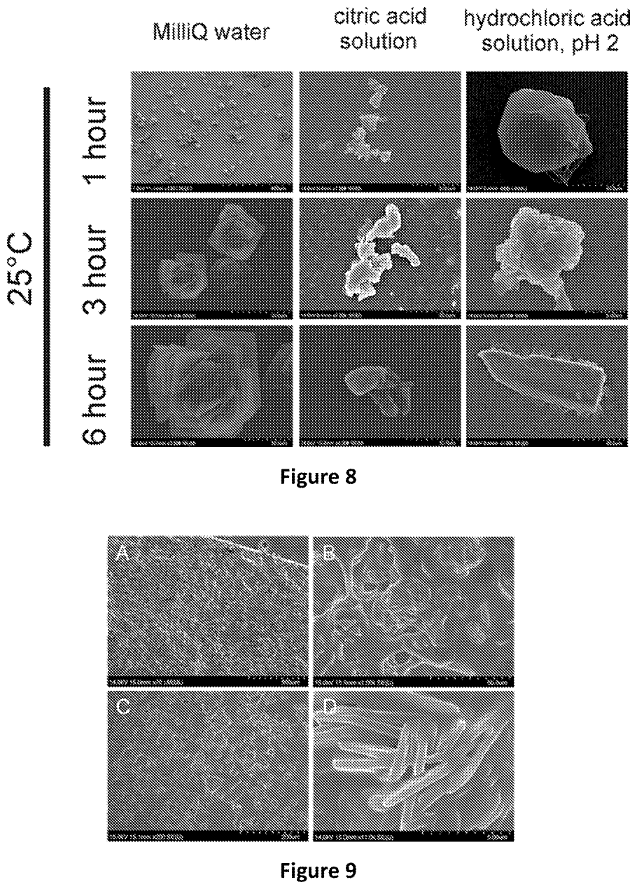

FIG. 8 examines the effect of water-based solvents having neutral and acidic pH on the structure and size of epinephrine base particles. FIG. 8 shows the structure of recrystallized epinephrine particles after being dissolved in selected water-based solvents (MiiliQ water, citric acid solution, and water solution acidified using HCl to pH 2). Nanostructured morphology is clearly missing in all of the observed particles of epinephrine, which are obtained as a result of recrystallization that occurs during the evaporation of the solvent/dispersant in preparation for electron microscopy.

FIG. 9 shows epinephrine base particle-containing film layers observed using SEM. The presence of epinephrine base solid particles in the formulated final film layer was confirmed, as shown in the SEM scans. The characteristic structure and size of epinephrine base particles was maintained in the final film layer, thus confirming no dissolution and recrystallization of epinephrine base particles occurred during the process of manufacturing the films. In particular, FIG. 9A shows epinephrine base particle-containing film layer manufactured using a solution of citric acid in acetone as a solvent/dispersant. FIG. 9B shows enlarged detail of film of 9A clearly confirming the preserved size and structure of the original epinephrine particles. FIG. 9C shows epinephrine base particle-containing film layer manufactured using ethanol with sodium deoxycholate as a solvent/dispersant. FIG. 9D shows enlarged detail of film of 9C, clearly confirming that the size and structure of original epinephrine particles is maintained.

### Conclusion

The size and structure of epinephrine particles is maintained when using acetone, ethanol, methanol, isopropyl alcohol, and alkaline water-based solvents/dispersants at temperatures of 25° C. and 45° C. for at least 6 hours. Small micron-sized nanostructured crystals were observed that existed before and after dispersion with the selected solvents/dispersants; these particles were not formed due to a process of recrystallization. The concentration of epinephrine base particles in the dispersant/solvents had no influence on the physical size and structure of the processed epinephrine base particles.

### Example 2

### Preparation of the Oromucosal Film Compositions of the Invention

Various formulations were designed including single, bi and tri-layered oromucosal films to demonstrate the use of epinephrine base particles in alternative formulation designs. Selected solvents/dispersants as listed in Example 1 were used in the formulation of the epinephrine base particle-containing layer.

### The Preparation Method

Each layer was formed separately (in the case of bi- and tri-layered films) and then laminated/combined to form the desired oromucosal film compositions. Alternatively, the layers could be built up in succession using the solvent casting or flexographic printing technique.

Each tri-layered film consists of 1) a backing layer, 2) a mucoadhesive layer and 3) an epinephrine particle-containing layer. In the first step the upper surface of a prepared mucoadhesive layer was coated with a polymer to form: i) a non-soluble backing layer (ethyl cellulose (Eleftheriadis et al. Fabrication of Mucoadhesive Buccal Films for Local Administration of Ketoprofen and Lidocaine Hydrochloride by Combining Fused Deposition Modeling and Inkjet Printing. J Pharm Sci. 2020 September; 109(9):2757-2766. doi: 10.1016/j.xphs.2020.05.022) or polycaprolactone (Colley et al. Pre-clinical evaluation of novel mucoadhesive bilayer patches for local delivery of clobetasol-17-propionate to the oral mucosa. Biomaterials. 2018 September; 178:134-146. doi: 10.1016/j.biomaterials.2018.06.009, Edmans et al. Mucoadhesive Electrospun Fiber-Based Technologies for Oral Medicine. Pharmaceutics. 2020 Jun. 2; 12(6):504. doi: 10.3390/pharmaceutics12060504); or ii) soluble backing layer (e.g. amino methacrylate copolymer-Eudragit® (Mašek et al. Multi-layered nanofibrous mucoadhesive films for buccal and sublingual administration of drug-delivery and vaccination nanoparticles—important step towards effective mucosal vaccines. J Control Release. 2017 Mar. 10; 249:183-195. doi: 10.1016/j.jconrel.2016.07.036)). In the second step, the bottom side of each mucoadhesive layer was moistened with a vapor stream and the epinephrine base particle-containing layer (third layer) was immediately pressed against this moistened mucoadhesive layer surface to form the tri-layered oromucosal film. Thermal-induced lamination could also be employed to attach the epinephrine containing third layer.

A variety of polymers, mucoadhesive polymers, and pharmaceutical excipients, including pH modifiers, absorption enhancers and antioxidants could be used in the formulations. In the case of pH modifiers, citric acid was used but other organic weak acids including tartaric, ascorbic and malic acids were also tested and could be used as alternatives to citric acid, e.g., adipic acid, ascorbic acid, citric acid, fumaric acid, glutaric acid, itaconic acid, maleic acid, malic acid, succinic acid, tartaric acid. Many other alternative absorption enhancers, plasticizers or antioxidants etc. could be used.

The non-adhesive backing layer was formed by i) spraying a 2.5% ethanolic solution of ethyl cellulose (Ethocel™, Colorcon Limited, UK) with 2% dibutyl phthalate (DBP, Merck KGaA, Germany) directly onto the surface of the mucoadhesive layer or the epinephrine base particle-containing layer, or ii) by spraying an ethanolic solution of amino methacrylate copolymer (Eudragit® L100-55, Evonik, Germany) to form an oro-dissolving backing layer. (Mašek et al. Multi-layered nanofibrous mucoadhesive films for buccal and sublingual administration of drug-delivery and vaccination nanoparticles—important step towards effective mucosal vaccines. J Control Release. 2017 Mar. 10; 249:183-195. doi: 10.1016/j.jconrel.2016.07.036). The non-adhesive backing layer was alternatively formed using solvent casting methodology and laminated to the mucoadhesive layer or the epinephrine base particle-containing layer.

Mucoadhesive layers were prepared by solvent casting. Carbomer (Carbopol® 974P, Lubrizol Advanced Materials, USA) and hypromellose 2208 (hydroxypropyl methylcellulose, viscosity 4000 mPa·s, Methocel™ K4M, Colorcon Limited, UK) were combined in a 2:1 (w/w) ratio in water to form a viscous opaque solution. Glycerol at 15% (w/w) was added as a plasticizer and the combined mixture was then treated by sonication to remove air bubbles. The required volume was then poured into plastic Petri dishes. The excess water was removed by evaporation at a temperature of 37° C. to produce the desired mucoadhesive layers. It is noted that the mucoadhesive layer could contain other excipients, for example to modify pH or enhance absorption. Table I describes composition of the mucoadhesive layers:

TABLE I
Mucoadhesive layer prepared by solvent casting method (polymer
swelling/dissolution conditions: 72 hrs at 25° C.; solvent
evaporation: 48 hrs at 37° C.) using water 0.592 ml/cm²:

| Material | % of dry composition | mg/cm² film |
|---|---|---|
| Carbomer | 56.070 | 4.734 |
| Hypromellose 2208 | 28.035 | 2.367 |
| Glycerol | 15.896 | 1.342 |
| Total | 100.000 | 8.443 |

Epinephrine base particle-containing layers were prepared using selected solvents/dispersants (see example 1) and polymers:

Formula I: Hypromellose 2910 (hydroxypropyl methylcellulose, viscosity 6 mPa·s, Pharmacoat® 606, Shin-Etsu Chemical Co. Ltd., Japan) and hydroxypropyl cellulose (viscosity 300-600 mPa·s, Klucel™ EF PHARM, Ashland, USA) in a 2:1 (w/w) ratio were added to the chosen solvent/disperant (ethanol, acetone, alkaline water of pH above 8.5 or mixture of thereof). Polyethylene glycol (PEG 600, Merck KGaA, Germany) at a concentration range of 12-17% (w/w) was added as a plasticizer. Sodium metabisulfite (Merck KGaA, Germany) in the concentration range of 0.007-2.284% was added as an antioxidant to stabilize the solution. Epinephrine base particles (Cambrex Profarmaco Milano S.r.l., Italy) were added at a concentration of 13.7-28.5% (w/w). Citric acid (Merck KGaA, Germany), ascorbic acid (Merck KGaA, Germany) and tartaric acid (Merck KGaA, Germany) was added to the mixture in the concentration range of 1.76-15.14% (w/w). Edetic acid (EDTA, Merck KGaA, Germany), sodium deoxycholate (Merck KGaA, Germany), polyoxyethylene (23) lauryl ether (Brij 35®, Merck KGaA, Germany), caprylocaproyl polyoxyl-8 glycerides NF (Labrasol®, Gattefossé, France) were added as absorption enhancers (2.5-2.9% w/w). The wet mixtures were then treated by sonication to remove air bubbles and the required volumes poured out into Petri dishes. The selected solvent/dispersant was then removed by evaporation at a temperature of 37° C. (ethanol, alkaline water of pH above 8.5) or at room temperature (acetone) to form the desired epinephrine base particle-containing layer. Each epinephrine base particle-containing oromucosal film layer contained 1.42-3.98 mg/cm² of epinephrine base.

The following tables describes examples of the composition of the epinephrine base particle-containing layers:

TABLE II
Epinephrine base particle-containing layer containing 18% of
epinephrine base (1.99-3.98 mg/cm²) prepared by solvent casting
method ((polymer swelling/dissolution conditions: 2 hrs at 25°
C.; solvent evaporation: 3 hrs at 37° C.) using ethanol or ethanol/alkaline
water mixture (80/20% v/v) 0.398 ml/cm²:

| Material | % of dry composition | mg/cm² film |
|---|---|---|
| Epinephrine base | 18.019 | 1.988 |
| Hypromellose 2910 | 44.446 | 4.904 |
| Hydroxypropyl Cellulose | 22.223 | 2.452 |
| Sodium Metabisulfite | 0.042 | 0.005 |
| Polyethylene Glycol | 15.271 | 1.685 |
| Total | 100.000 | 11.033 |
| Epinephrine base | 17.839 | 2.982 |
| Hypromellose 2910 | 43.608 | 7.290 |
| Hydroxypropyl Cellulose | 21.804 | 3.645 |
| Sodium Metabisulfite | 1.070 | 0.179 |
| Polyethylene Glycol | 15.679 | 2.621 |
| Total | 100.000 | 16.716 |
| Epinephrine base | 17.858 | 3.976 |
| Hypromellose 2910 | 44.644 | 9.940 |
| Hydroxypropyl Cellulose | 22.322 | 4.970 |
| Sodium Metabisulfite | 0.042 | 0.009 |
| Polyethylene Glycol | 15.134 | 3.370 |
| Total | 100.000 | 22.266 |

TABLE IIIa
Examples of the preparation epinephrine base particle-containing
layer with concentration of epinephrine base in the range
of 13.7-22.6% prepared by solvent casting method (polymer
swelling/dissolution conditions: 2 hrs at 25° C.; solvent
evaporation 3 hrs at 37° C.) using ethanol 0.398
ml/cm² or ethanol/alkaline water mixture (80/20% v/v):

| Material | % of dry composition | mg/cm² film |
|---|---|---|
| Epinephrine base | 13.705 | 1.420 |
| Hypromellose 2910 | 45.683 | 4.734 |
| Hydroxypropyl Cellulose | 22.841 | 2.367 |
| Sodium Metabisulfite | 2.284 | 0.237 |
| Polyethylene Glycol | 15.487 | 1.605 |
| Total | 100.000 | 10.362 |
| Epinephrine base | 22.623 | 2.982 |
| Hypromellose 2910 | 40.218 | 5.302 |
| Hydroxypropyl Cellulose | 20.109 | 2.651 |
| Sodium Metabisulfite | 0.007 | 0.001 |
| Polyethylene Glycol | 17.043 | 2.247 |
| Total | 100.000 | 13.182 |

TABLE IIIb
Examples of the preparation epinephrine base particle-containing
layer with concentration of epinephrine base in range 14-
24.6% prepared by solvent casting method (solvent evaporation 2
hrs at 25° C.) using acetone 0.393 ml/cm².

| Material | % of dry composition | mg/cm² film |
|---|---|---|
| Epinephrine base | 14.025 | 1.887 |
| Hypromellose 2910 | 46.751 | 6.290 |
| Hydroxypropyl Cellulose | 23.375 | 3.145 |
| Sodium Metabisulfite | 0.000 | 0.000 |
| Polyethylene Glycol | 15.848 | 2.132 |
| Total | 100.000 | 13.455 |
| Epinephrine base | 24.600 | 3.774 |
| Hypromellose 2910 | 41.001 | 6.290 |
| Hydroxypropyl Cellulose | 20.500 | 3.145 |
| Sodium Metabisulfite | 0.000 | 0.000 |
| Polyethylene Glycol | 13.899 | 2.132 |
| Total | 100.000 | 15.342 |

TABLE IIIc
Examples of the preparation micron-size epinephrine base particle-
containing layer containing 1.66, 1.99, 2.99 mg/cm² micronized
epinephrine base in the range of 17.9-19.5% prepared by solvent
casting method (polymer swelling/dissolution conditions: 2 hrs at
25° C.; solvent evaporation: 3 hrs at 37° C.) using ethanol/alkaline
water mixture (80/20% v/v) or ethanol/alkaline water mixture (90/10%
v/v) 0.398 ml/cm²:

| Material | % of dry composition | mg/cm² film |
|---|---|---|
| Epinephrine base | 19.463 | 1.657 |
| Hypromellose 2910 | 42.818 | 3.645 |
| Hydroxypropyl Cellulose | 21.409 | 1.822 |
| Sodium Metabisulfite | 1.051 | 0.089 |
| Polyethylene Glycol | 15.259 | 1.299 |
| Total | 100.000 | 8.512 |
| Epinephrine base | 17.898 | 1.988 |
| Hypromellose 2910 | 43.551 | 4.838 |
| Hydroxypropyl Cellulose | 21.775 | 2.419 |
| Sodium Metabisulfite | 1.074 | 0.119 |
| Polyethylene Glycol | 15.702 | 1.744 |
| Total | 100.000 | 11.108 |
| Epinephrine base | 17.864 | 2.982 |
| Hypromellose 2910 | 43.668 | 7.290 |
| Hydroxypropyl Cellulose | 21.834 | 3.645 |
| Sodium Metabisulfite | 1.072 | 0.179 |
| Polyethylene Glycol | 15.562 | 2.598 |
| Total | 100.000 | 16.693 |

The following tables describes the composition of the epinephrine base particle-containing layers in combination with other excipients:

TABLE IVa
Examples of preparation of epinephrine base particle-
containing layers with pH modifier (citric acid) prepared
by solvent casting method (solvent evaporation: 2 hrs at
25° C.) using acetone 0.398 ml/cm²:

| | | |
|---|---|---|
| Material | % of dry composition | mg/cm² film |

Epinephrine base: citric acid 1:1

| | | |
|---|---|---|
| Epinephrine base | 15.135 | 2.982 |
| Hypromellose 2910 | 37.024 | 7.290 |
| Hydroxypropyl Cellulose | 18.487 | 3.645 |
| Sodium Metabisulfite | 0.914 | 0.179 |
| Polyethylene Glycol | 13.306 | 2.621 |
| Citric Acid | 15.135 | 2.982 |
| Total | 100.000 | 19.698 |

Epinephrine base: citric acid 10:1

| | | |
|---|---|---|
| Epinephrine base | 17.519 | 2.982 |
| Hypromellose 2910 | 42.857 | 7.290 |
| Hydroxypropyl Cellulose | 21.399 | 3.645 |
| Sodium Metabisulfite | 1.058 | 0.179 |
| Polyethylene Glycol | 15.403 | 2.621 |
| Citric Acid | 1.764 | 0.298 |
| Total | 100.000 | 17.015 |

TABLE IVb
Examples of the preparation of epinephrine base particle-
containing layers with absorption enhancers (sodium deoxycholate,
edetic acid) prepared by solvent casting method (polymer
swelling/dissolution conditions: 2 hrs at 25° C.; solvent
evaporation: 3 hrs at 37° C.) using ethanol 0.398
ml/cm² or ethanol/alkaline water mixture (80/20% v/v):

| Material | % of dry composition | mg/cm² film |
|---|---|---|
| Epinephrine base | 17.316 | 2.982 |
| Hypromellose 2910 | 42.359 | 7.290 |
| Hydroxypropyl Cellulose | 21.150 | 3.645 |
| Sodium Metabisulfite | 1.046 | 0.179 |
| Polyethylene Glycol | 15.224 | 2.621 |
| Sodium Deoxycholate | 2.905 | 0.497 |
| Total | 100.000 | 17.213 |
| Epinephrine base | 17.316 | 2.982 |
| Hypromellose 2910 | 42.359 | 7.290 |
| Hydroxypropyl Cellulose | 21.150 | 3.645 |
| Sodium Metabisulfite | 1.046 | 0.179 |
| Polyethylene Glycol | 15.224 | 2.621 |
| Edetic acid | 2.905 | 0.497 |
| Total | 100.000 | 17.213 |

TABLE IVc
Examples of the preparation of epinephrine base particle-containing
layers with pH modifier (citric acid or tartaric acid or ascorbic
acid) and absorption enhancers (sodium deoxycholate, edetic acid,
polyoxyethylene lauryl ether - Brij 35 ®, caprylocaproyl polyoxyl-8
glycerides NF - Labrasol ®) prepared by solvent casting method (polymer
swelling/dissolution conditions: 2 hrs at 25° C.; solvent evaporation:
3 hrs at 37° C.) using ethanol or acetone 0.398 ml/cm²:

| Material | % of dry composition | mg/cm² film |
|---|---|---|
| Epinephrine base | 14.760 | 2.982 |
| Hypromellose 2910 | 36.107 | 7.290 |
| Hydroxypropyl Cellulose | 18.029 | 3.645 |
| Sodium Metabisulfite | 0.892 | 0.179 |
| Polyethylene Glycol | 12.977 | 2.621 |
| Citric Acid | 14.760 | 2.982 |
| Sodium Deoxycholate | 2.476 | 0.497 |
| Total | 100.000 | 20.195 |
| Epinephrine base | 14.760 | 2.982 |
| Hypromellose 2910 | 36.107 | 7.290 |
| Hydroxypropyl Cellulose | 18.029 | 3.645 |
| Sodium Metabisulfite | 0.892 | 0.179 |
| Polyethylene Glycol | 12.977 | 2.621 |
| Citric Acid | 14.760 | 2.982 |
| Edetic acid | 2.476 | 0.497 |
| Total | 100.000 | 20.195 |
| Epinephrine base | 15.943 | 2.982 |
| Hypromellose 2910 | 38.973 | 7.290 |
| Hydroxypropyl Cellulose | 19.486 | 3.645 |
| Sodium Metabisulfite | 0.957 | 0.179 |
| Polyethylene Glycol | 14.012 | 2.621 |
| Tartaric Acid | 7.972 | 1.491 |
| Polyoxyethylene lauryl ether | 2.657 | 0.497 |
| Total | 100.000 | 18.704 |
| Epinephrine base | 16.744 | 2.982 |
| Hypromellose 2910 | 40.930 | 7.290 |
| Hydroxypropyl Cellulose | 20.465 | 3.645 |
| Sodium Metabisulfite | 1.005 | 0.179 |
| Polyethylene Glycol | 14.716 | 2.621 |
| Ascorbic Acid | 3.349 | 0.596 |
| Labrasol | 2.791 | 0.497 |
| Total | 100.000 | 17.810 |

Formula II: Epinephrine base particle-containing layer containing only hypromellose 2910 (hydroxypropyl methylcellulose, viscosity 6 mPa s, Pharmacoat® 606, Shin-Etsu Chemical Co. Ltd., Japan) was prepared by solvent casting method.

TABLE Va
Epinephrine base particle-containing layer with 18% of epinephrine
base prepared by solvent casting method (polymer swelling/dissolution
conditions: 2 hrs at 25° C.; solvent evaporation: 3 hrs at
37° C.) using ethanol or ethanol/alkaline water mixture (80/20%
v/v) 0.398 ml/cm²:

| Material | % of dry composition | mg/cm² film |
|---|---|---|
| Epinephrine base | 18.080 | 2.982 |
| Hypromellose 2910 | 66.292 | 10.934 |
| Sodium Metabisulfite | 2.009 | 0.331 |
| Polyethylene Glycol | 13.620 | 2.247 |
| Total | 100.000 | 16.494 |

TABLE Vb
Epinephrine base particle-containing layer with 28% of epinephrine
base prepared by solvent casting method (polymer swelling/dissolution
conditions: 2 hrs at 25° C.; solvent evaporation: 6 hrs at
37° C.) using ethanol or ethanol/alkaline water mixture (80/20%
v/v) 0.469 ml/cm²:

| Material | % of dry composition | mg/cm² film |
|---|---|---|
| Epinephrine base | 28.511 | 3.756 |
| Hypromellose 2910 | 59.399 | 7.825 |
| Sodium Metabisulfite | 0.009 | 0.001 |
| Polyethylene Glycol | 12.082 | 1.592 |
| Total | 100.000 | 13.173 |

Formula III: Epinephrine base particle-containing layer with hypromellose 2910 (hydroxypropyl methylcellulose, viscosity 6 mPa s, Pharmacoat® 606, Shin-Etsu Chemical Co. Ltd., Japan) and hypromellose 2208 (hydroxypropyl methylcellulose, viscosity 4000 mPa·s, Methocel™ K4M, Colorcon Limited, UK) was prepared by solvent casting method (polymer swelling/dissolution conditions: 2 hrs at 25° C.; solvent evaporation: 6 hrs at 37° C.) using ethanol or ethanol/alkaline water mixture (80/20% v/v) 0.592 ml/cm² as described above for Formula I.

TABLE VI

| Material | % of dry composition | mg/cm² film |
|---|---|---|
| Epinephrine base | 13.705 | 1.420 |
| Hypromellose 2910 | 45.683 | 4.734 |
| Hypromellose 2208 | 22.841 | 2.367 |
| Sodium Metabisulfite | 2.284 | 0.237 |
| Polyethylene Glycol | 15.487 | 1.605 |
| Total | 100.000 | 10.362 |

Formula IV: Epinephrine base particle-containing layer with hypromellose 2910 (hydroxypropyl methylcellulose, viscosity 6 mPa s, Pharmacoat® 606, Shin-Etsu Chemical Co. Ltd., Japan) and Povidon (Polyvinylpyrrolidone K 30, Merck KGaA, Germany) was prepared by solvent casting method (polymer swelling/dissolution conditions: 2 hrs at 25° C.; solvent evaporation: 3 hrs at 37°) using ethanol or ethanol/alkaline water mixture (80/20% v/v) 0.592 ml/cm².

TABLE VII

| Material | % of dry composition | mg/cm² film |
|---|---|---|
| Epinephrine base | 13.705 | 1.420 |
| Hypromellose 2910 | 45.683 | 4.734 |
| Povidon | 22.841 | 2.367 |
| Sodium Metabisulfite | 2.284 | 0.237 |
| Polyethylene Glycol | 15.487 | 1.605 |
| Total | 100.000 | 10.362 |

### Example 3

### Dissolution of Epinephrine Base Particles Formulated as an Oromucosal Film/Polymeric Layer of the Invention

This dissolution study was performed to confirm the effect of pH modifiers and absorption enhancers on the dissolution rate of epinephrine base particles contained in oromucosal films of the invention. Epinephrine base particles formulated in an oromucosal film of the invention dissolve quickly and are released from the oromucosal film within minutes. Rapid release of epinephrine following in-vivo administration in patients suffering from anaphylactic shock is a key requirement for any anaphylaxis treatment.

The following oromucosal films were studied for dissolution rate and solubility with and without a pH modifier or absorption enhancer:
i) Epinephrine base particles formulated in an oromucosal film.
ii) Epinephrine base particles formulated in an oromucosal film with citric acid as a pH modifier.
iii) Epinephrine base particles formulated in an oromucosal film with sodium deoxycholate as an absorption enhancer.

The standard USP dissolution test was modified to clearly show the effect of an acidifier (pH modifier) and an absorption enhancer on both the dissolution rate and solubility as follows:

The dissolution test was performed using either simulated saliva (pH 6.8) or citric acid solution (15 mg/ml) as the dissolution medium with 30 mg/ml sodium metabisulfite as antioxidant maintained at 37±0.5° C. The total volume used in all cases was 20 ml. The solutions were continually mixed using a magnetic stirrer (100 rpm) during the whole experiment. The oromucosal film was cut into a square of 1 cm² and adhered to a glass plate held in position with double sided adhesive tape. 0.5 ml samples were withdrawn at time intervals of 0, 1, 2, 3, 4, 5, 10, 15 and 60 minutes with subsequent addition of 5 μl of 10% solution of formic acid into each sample. As each sample was withdrawn, it was replaced with the same volume of fresh medium. The concentration of epinephrine in each of the samples was measured by using liquid chromatography—tandem mass spectrometry (LCMS/MS).

### Sample Preparation for LCMS/MS

200 μl of dissolution medium was mixed with 100 μl (±)-epinephrine-D6 internal standard (concentration 10 μg/ml) and 1000 μl 2-propanol. The sample was vortexed and left at 6° C. for 30 minutes. The sample was then centrifuged for 30 minutes at 10 000 rpm at 4° C. After centrifugation extracts were dried under a stream of nitrogen, redissolved in 150 μl of 50% methanol with 0.1% formic acid and an aliquot of 5 μl was injected into the HPLC column.

### LCMS/MS

Sample analyses were performed by using liquid chromatography-tandem mass spectrometry. An Agilent 1200 chromatographic system (Agilent Technologies, Germany), consisting of binary pump, vacuum degasser, autosampler and thermostatted column compartment, was used. Separation of epinephrine was carried out using an Ascentis Express C18, 2.1×150 mm, 2.7 μm particle size column (Supelco, Bellefonte, Pa., USA) with a 20-min linear gradient from 50 to 100% of methanol. Mobile phase contained 0.1% of formic acid. The flow rate of the mobile phase was 0.2 ml/min, the column temperature was set at 45° C. A triple quadrupole mass spectrometer Agilent 6410 Triple Quad LCMS (Agilent Technologies, USA) with an electrospray interface (ESI) was used for detection of the analyte. Epinephrine (MRM transition m/z 166−57) and internal standard (172−157) were detected using multi reaction monitoring mode.

The standard USP dissolution test was modified to simulate the effect of a limited amount of fluid following administration of the film to oral mucosa in vivo; lowering the total volume in the dissolution test better mimics in-vivo conditions. The 20 ml volume of dissolution media was considered as a good compromise between both the standard USP dissolution test and the real in-vivo conditions occurring in the oral mucosa. This volume was also considered appropriate to demonstrate the effect of citric acid as a pH modifier and the effect of deoxycholate sodium as a absorption enhancer.

The following formulations of epinephrine base particles as oromucosal films were tested:
1) The composition of the film layer containing epinephrine base particles

| Material | % of dry composition | mg/cm² film |
|---|---|---|
| Epinephrine base | 17.83 | 2.98 |
| Pharmacoat 606 | 43.63 | 7.29 |
| Klucel EF | 21.78 | 3.64 |
| Sodium metabisulfite | 1.08 | 0.18 |
| PEG 600 | 15.68 | 2.62 |
| Total | 100.00 | 16.71 |

2) The composition of the oromucosal films containing epinephrine base particles co-formulated with a pH modifier (citric acid)

| Material | % of dry composition | mg/cm² film |
|---|---|---|
| Epinephrine base | 15.13 | 2.98 |
| Pharmacoat 606 | 37.02 | 7.29 |
| Klucel EF | 18.49 | 3.64 |
| Sodium metabisulfite | 0.91 | 0.18 |
| PEG 600 | 13.31 | 2.62 |
| Citric acid | 15.13 | 2.98 |
| Total | 100.00 | 19.69 |

3) The composition of the oromucosal film layer containing epinephrine base particles co-formulated with an absorption enhancer (sodium deoxycholate)

| Material | % of dry composition | mg/cm² film |
|---|---|---|
| Epinephrine base | 17.32 | 2.98 |
| Pharmacoat 606 | 42.36 | 7.29 |
| Klucel EF | 21.15 | 3.64 |
| Sodium metabisulfite | 1.05 | 0.18 |
| PEG 600 | 15.22 | 2.62 |
| Sodium deoxycholate | 2.91 | 0.5 |
| Total | 100.00 | 17.21 |

The most significant difference in dissolution profile is seen when comparing the epinephrine base particle-containing film formulated with citric acid and when the dissolution test was performed in citric acid solution as a dissolution medium. These conditions help in understanding the behavior of epinephrine base particles formulated as an oromucosal film administered to oral mucosa with the presence of limited amounts of fluid. The results show that citric acid solution has a significantly positive effect on the dissolution rate and solubility of epinephrine base particles. The artificial saliva solution (pH 6.8) showed no significant impact on dissolution rate or solubility. To conclude, the presence of pH modifiers (acidifiers), as demonstrated by the example of citric acid here, is beneficial for the dissolution rate and increased local solubility of epinephrine base particles.

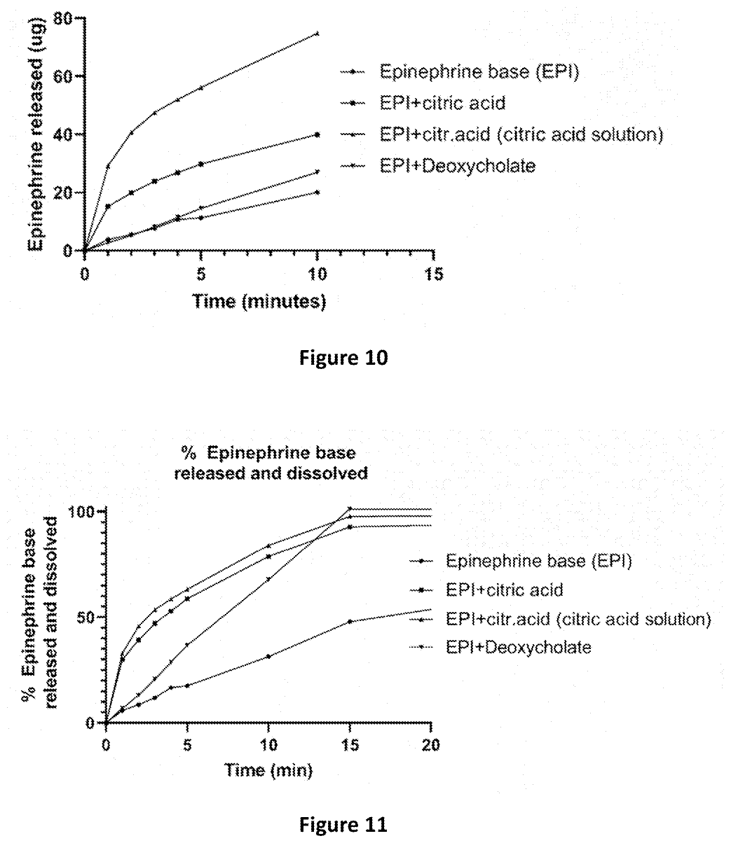

FIG. 10 shows the effect of absorption enhancers and pH modifiers on the dissolution rate and solubility of epinephrine base particles in the dissolution medium. The effect of micro-environmental pH on dissolution rate of epinephrine base particles is clearly shown when comparing the dissolution profile of epinephrine base, epinephrine base particles co-formulated with citric acid and epinephrine base particles co-formulated with citric acid with the dissolution test performed in a citric acid dissolution medium. The highest level of epinephrine base particle dissolution rate and solubility was shown when it was formulated with citric acid and when the dissolution test was performed in citric acid solution as the dissolution medium.

FIG. 11 shows the amount of dissolved Epinephrine base particles expressed in percentage. For reference, the dissolved and released amount of epinephrine from oromucosal film after 1 hour was considered as 100% in the graph. Although, there is improved solubility of epinephrine base particles when a citric acid dissolution medium is selected, the graph clearly shows the greater beneficial effect of co-formulation with a pH acidifier (citric acid) on the dissolution profile of epinephrine base particles.

### Example 4

### Ex-Vivo Absorption of Epinephrine Base Particles Formulated in an Oromucosal Film of the Invention

The objective of this study was to study the effect of absorption enhancers on the absorption rate of epinephrine base solid particles formulated as an oromucosal film according to the present invention following the application (administration) of the film onto excised sublingual porcine mucosa ex-vivo using a diffusion cell model.

### Methods

Porcine sublingual mucosa was removed immediately after sacrifice. The mucosa was then prepared and mounted to the diffusion cell with the receptor compartment having a volume of 2 mL, containing both citric acid solution (3 mg/ml) and sodium metabisulfite (30 mg/ml). The oromucosal film (1 cm², 2.98 mg of epinephrine base) formulation of epinephrine base particles was then applied to the surface of the sublingual mucosa and moistened with 50 ul of artificial saliva solution (buffer, pH 6.8). The rate of absorption was assessed by measuring the increasing concentration of epinephrine in the receptor compartment under constant mixing at 37° C. Samples were taken at 0, 1, 3, 5, 8, 11, 14, 17 and 20 minutes, added 5 μl 10% solution of formic acid, to measure the concentration of epinephrine in the receptor chamber. The concentration level was determined as described in Example 3.

### Sample Preparation for LCMS/MS

200 μl of fluid from receptor chamber was mixed with 100 μl (±)-epinephrine-D6 internal standard (concentration 10 μg/ml) and 1000 μl 2-propanol. The sample was vortexed and left at 6° C. for 30 minutes. The sample was then centrifuged for 30 minutes at 10000 rpm at 4° C. After centrifugation extracts were dried under a stream of nitrogen, redissolved in 150 μl of 50% methanol with 0.1% formic acid and an aliquot of 5 μl was injected into the HPLC column.

The following formulations of epinephrine base particles as oromucosal films were tested:
1) The composition of the oromucosal film layer containing epinephrine base particles

| Material | % of dry composition | mg/cm² film |
|---|---|---|
| Epinephrine base | 17.83 | 2.98 |
| Pharmacoat 606 | 43.63 | 7.29 |
| Klucel EF | 21.78 | 3.64 |
| Sodium metabisulfite | 1.08 | 0.18 |
| PEG 600 | 15.68 | 2.62 |
| Total | 100.00 | 16.71 |

2) The composition of the oromucosal film layer containing epinephrine base particles co-formulated with a absorption enhancer (sodium deoxycholate)

| Material | % of dry composition | mg/cm² film |
|---|---|---|
| Epinephrine base | 17.32 | 2.98 |
| Pharmacoat 606 | 42.36 | 7.29 |
| Klucel EF | 21.15 | 3.64 |
| Sodium metabisulfite | 1.05 | 0.18 |
| PEG 600 | 15.22 | 2.62 |
| Sodium deoxycholate | 2.91 | 0.5 |
| Total | 100.00 | 17.21 |

3) The composition of the oromucosal film layer containing epinephrine base particles co-formulated with pH modifier (citric acid) and absorption enhancer (edetic acid)

| Material | % of dry composition | mg/cm² film |
|---|---|---|
| Epinephrine base | 14.760 | 2.982 |
| Hypromellose 2910 | 36.107 | 7.290 |
| Hydroxypropyl Cellulose | 18.029 | 3.645 |
| Sodium Metabisulfite | 0.892 | 0.179 |
| Polyethylene Glycol | 12.977 | 2.621 |
| Citric acid | 14.760 | 2.982 |
| Edetic acid | 2.476 | 0.497 |
| Total | 100.000 | 20.195 |

### Results

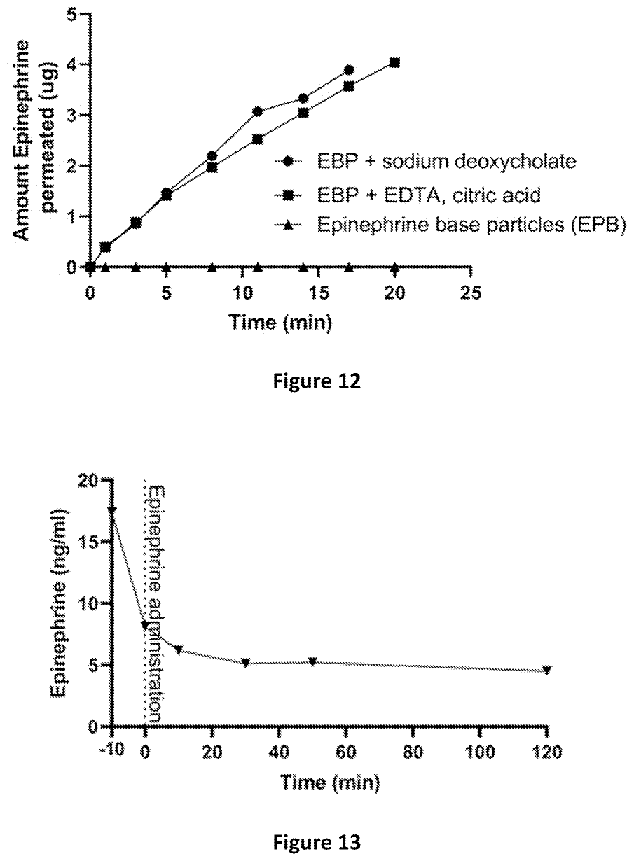

FIG. 12 shows the influence of absorption enhancers and pH modifiers on the absorption rate of epinephrine directly released from an oromucosal film with epinephrine base solid particles using a diffusion cell.

Surprisingly, the absorption of epinephrine through sublingual mucosa ex-vivo was observed within a minute of the administration of the oromucosal film formulation of the invention comprising epinephrine base particles. The absorption of significant levels of epinephrine occurred within 1-3 minutes following administration of the film. The level of epinephrine was quantifiable after the first minute, with only a very slight lag followed by a linear increase in permeated epinephrine (FIG. 12) over 20 minutes.

### Conclusion

Surprisingly, epinephrine in the form of solid epinephrine base particles formulated in an oromucosal film of the invention is able to permeate within 1-3 minutes from when it is applied to sublingual porcine mucosa ex-vivo. It was also demonstrated that absorption enhancers significantly increase the absorption rate of epinephrine, even though it is formulated as solid particles (in the oromucosal film) and hence needs to dissolve directly at the site of administration.

### Example 5

### Pharmacokinetic Study of Epinephrine Base Particles Formulated in an Oromucosal Film of the Invention

The pharmacokinetic profile of epinephrine base particles formulated in an oromucosal film of the invention was compared to an intramuscular injection of epinephrine using a pharmacokinetic model in piglets (Sus scrofa domesticus). Blood plasma concentrations of epinephrine were measured following administration of the formulations to the sublingual mucosa and muscle, respectively. The study compared EpiPen 0.3 mg (Mylan) with an epinephrine oromucosal film (8 mg) containing epinephrine base particles.

The oromucosal film was formulated as a tri-layered film. The following formulations of epinephrine base particles as oromucosal films were tested:

The composition of the film layer containing epinephrine base particles, for sublingual application two mucoadhesive films were applied simultaneously (2×4 mg of epinephrine base):

| Material | % of dry composition | mg/cm² film |
|---|---|---|
| Epinephrine base | 13.70 | 1.42 |
| Hypromellose 2910 | 45.68 | 4.73 |
| Hydroxypropyl Cellulose | 22.84 | 2.37 |
| Sodium Metabisulfite | 2.28 | 0.24 |
| Polyethylene Glycol | 15.49 | 1.61 |
| Total | 100.00 | 10.36 |

The oromucosal film was formulated as described in Example 2. The size of the film layer containing epinephrine base particles used in the experiment: 2.5 cm×1.1 cm.

Piglets weighing 18-20 Kg (n=3) were utilized. The piglets were anesthetized by intramuscular injection and plasma levels of endogenous epinephrine were monitored prior to epinephrine administration (sublingual or intramuscular). Samples of 200 μl of blood plasma were taken and epinephrine levels were measured using mass spectrometry.

### Sample Preparation for LCMS/MS

200 μl of plasma was mix with 100 μl (±)-Epinephrine-D6 internal standard (concentration 10 μg/ml) and 1000 μl 2-propanol. The sample was vortexed and left at 6° C. for 30 minutes. The sample was then centrifuged for 30 minutes at 10000 rpm at 4° C. After centrifugation extracts were dried under a stream of nitrogen, redissolved in 150 μl of 50% methanol with 0.1% formic acid and an aliquot of 5 μl was injected into the HPLC column. The concentration level was determined as described in Example 3.

The epinephrine formulations were administered to the animals only after endogenous levels of epinephrine decreased to less than 10 ng/ml; this took approximately 20 mins following anesthesia of the animals. The initial high levels of endogenous epinephrine probably occurred due to stress caused by the physical manipulation of the animals prior to anesthesia.

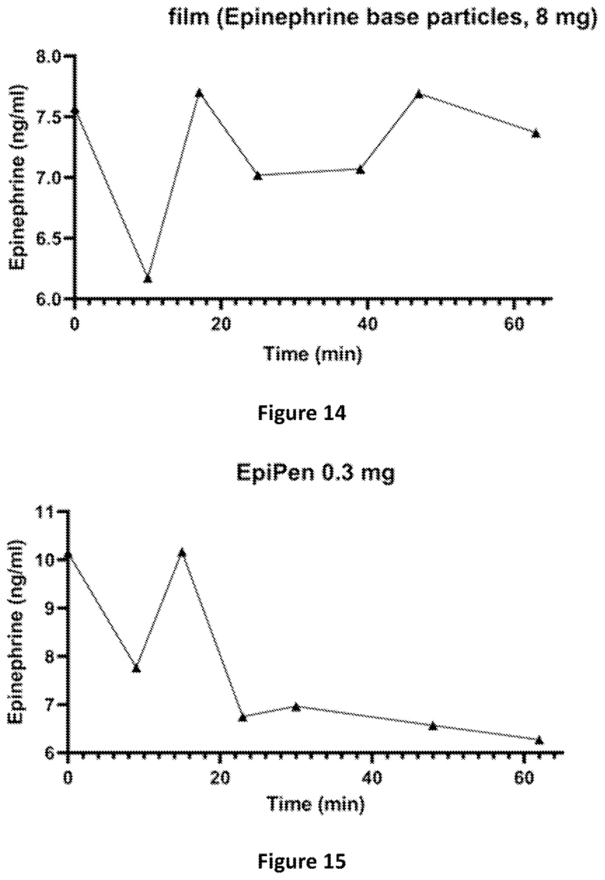

FIGS. 13-15 show plasma concentrations of epinephrine (ng/mL) as a function of time (minutes). FIG. 13 shows epinephrine concentration in blood plasma of piglets following administration of a control/placebo film. FIG. 14 shows concentration of epinephrine in blood plasma of piglets following sublingual administration of the oromucosal film composition of the invention containing 8 mg of epinephrine base particles. FIG. 15 shows concentration of epinephrine in blood plasma of piglets following EpiPen 0.3 mg administration.

The results show increased plasma concentrations of both sublingually administered epinephrine base particles formulated in an oromucosal film (FIG. 14) and 0.3 Epipen (Mylan) (FIG. 15). This is compared to the lower plasma levels of epinephrine in the control animal as shown in FIG. 13. This demonstrates that the epinephrine base solid particles formulated as an oromucosal film composition according to the present invention has a comparable pharmacokinetic profile to the EpiPen; the profile is especially similar in terms of Tₘₐₓ.

### Example 6

### Plasma Levels of Epinephrine Following the Administration of Epinephrine Base Particle-Containing Oromucosal Film of the Invention to Rabbits

To prove the efficacy of the oromucosal epinephrine base particle-containing film formulation of the invention, plasma concentration of epinephrine following sublingual administration was studied using a rabbit model. The study compares 6 mg epinephrine base particle-containing film and a placebo film.

The oromucosal film was formulated as a tri-layered film. Composition of the film layer containing epinephrine base particles for sublingual application—two mucoadhesive films were applied simultaneously (2×3 mg of epinephrine base):

| Material | % of dry composition | mg/cm² film |
|---|---|---|
| Epinephrine base | 17.839 | 2.982 |
| Hypromellose 2910 | 43.608 | 7.290 |
| Hydroxypropyl Cellulose | 21.804 | 3.645 |
| Sodium Metabisulfite | 1.070 | 0.179 |
| Polyethylene Glycol | 15.679 | 2.621 |
| Total | 100.000 | 16.716 |

The size of the film layer containing epinephrine base particles used in the experiment: 0.8 cm×1.3 cm.

New Zealand rabbits weighing 5-6 Kg were used in this experiment. The rabbits were anesthetized using an intramuscular injection of Ketamine and Xylazine. Epinephrine/placebo film was administered approximately 30 mins after the animals were anesthetized. The plasma level of epinephrine was determined 10 minutes prior to the sublingual administration of the oromucosal epinephrine base particle-containing film and once every 10 minutes for 60 minutes after administration. 200 μl samples of blood plasma were taken for analysis at each interval. Mass spectrometry and the same analytical protocol as described in Example 5 was utilized to determine plasma levels of epinephrine.

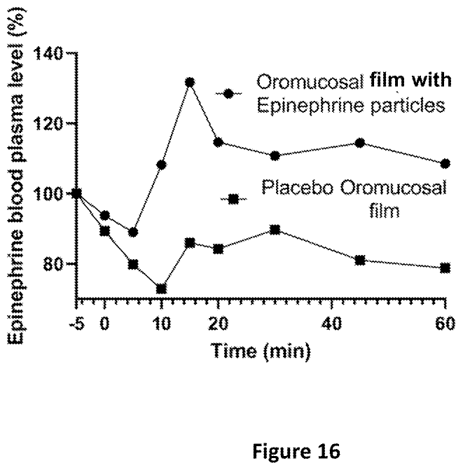

FIG. 16 shows the level of epinephrine in the blood samples following sublingual administration of oromucosal films; one placebo film and another formulated with epinephrine base particles according to the invention. The graph in FIG. 16 shows epinephrine plasma concentrations (expressed as percentage of the initial epinephrine plasma level) as a function of time in minutes. This example demonstrates that the epinephrine base solid particles formulated as an oromucosal film composition according to the present invention are efficacious in rabbit.

### Example 7

### Design of Mucoadhesive Films with pH Modifier Formulated in the Backing Layer and Effect of pH Modifier on Dissolution of Epinephrine Base Particles after Administration Ex Vivo

Each tested bi-layered film consists of 1) a backing layer, and 2) an Epinephrine base particle-containing mucoadhesive layer. The backing layer contains a pH modifier. Citric acid was used as an example of a pH modifier. Both layers, the non-adhesive backing layer, and mucoadhesive epinephrine particle-containing layers were prepared using the solvent casting method.
1) The Composition of the Backing Layer with Citric:

TABLE VIII
The backing layer prepared by solvent casting method (polymer
swelling/dissolution conditions: 3 hrs at 25° C.;
solvent evaporation: 4 hrs at 37° C. or 1 hrs at 60°
C.) using ethanol and water mixture (80/20% v/v) 0.331 ml/cm2:

| Material | % of dry composition | mg/film unit |
|---|---|---|
| Citric acid | 14.7 | 10.06 |
| Pharmacoat 606 | 46.8 | 31.93 |
| Klucel EF | 23.4 | 15.96 |
| PEG 600 | 15.1 | 10.28 |
| Total | 100.0 | — |
| Citric acid | 16.5 | 10.06 |
| Pharmacoat 606 | 52.5 | 31.93 |
| Klucel EF | 26.3 | 15.96 |
| Glycerol 99.7% | 4.7 | 2.84 |
| Total | 100.0 | — |
| Citric acid | 22.1 | 10.06 |
| Pharmacoat 606 | 48.6 | 22.09 |
| Klucel EF | 24.3 | 11.04 |
| Glycerol 99.7% | 5.0 | 2.30 |
| Total | 100.0 | — |

2) The Composition of the Mucoadhesive Film Layers Containing Epinephrine Base Particles:

TABLE IX
The mucoadhesive layer prepared by solvent casting method (polymer
swelling/dissolution conditions: 3 hrs at 25° C.; solvent
evaporation: 4 hrs at 37° C. or 1 hrs at 60° C.) using ethanol
and alkaline water mixture (80/20% v/v) 0.331 ml/cm²:

| | | |
|---|---|---|
| Material | % of dry composition | mg/film unit |

Composition 1A-2

| | | |
|---|---|---|
| Epinephrine base | 20.1 | 10.06 |
| Pharmacoat 606 | 41.5 | 20.78 |
| Klucel EF | 14.0 | 7.00 |
| Sodium Metabisulfite | 1.2 | 0.60 |
| Transcutol HP | 9.2 | 4.59 |
| PEG 600 | 14.0 | 7.00 |
| Total | 100.0 | — |

Composition 1B-2

| | | |
|---|---|---|
| Epinephrine base | 24.8 | 10.06 |
| Pharmacoat 606 | 51.1 | 20.78 |
| Klucel EF | 17.2 | 7.00 |
| Sodium Metabisulfite | 1.5 | 0.60 |
| Glycerol 99.7% | 5.4 | 2.19 |
| Total | 100.0 | — |

Composition 1C-2

| | | |
|---|---|---|
| Epinephrine base | 22.2 | 10.06 |
| Pharmacoat 606 | 45.9 | 20.78 |
| Klucel EF | 15.5 | 7.00 |
| Sodium Metabisulfite | 1.3 | 0.60 |
| Glycerol 99.7% | 4.8 | 2.19 |
| Transcutol HP | 10.2 | 4.59 |
| Total | 100.0 | — |

Composition 2A-2

| | | |
|---|---|---|
| Epinephrine base | 16.7 | 10.06 |
| Pharmacoat 606 | 45.7 | 27.55 |
| Klucel EF | 15.2 | 9.18 |
| Sodium Metabisulfite | 1.0 | 0.60 |
| PEG 600 | 13.8 | 8.31 |
| Transcutol HP | 7.6 | 4.59 |
| Total | 100.0 | — |

Composition 2B-2

| | | |
|---|---|---|
| Epinephrine base | 20.3 | 10.06 |
| Pharmacoat 606 | 55.6 | 27.55 |
| Klucel EF | 18.5 | 9.18 |
| Sodium Metabisulfite | 1.2 | 0.60 |
| Glycerol 99.7% | 4.4 | 2.19 |
| Total | 100.0 | — |

Composition 2C-2

| | | |
|---|---|---|
| Epinephrine base | 18.4 | 10.06 |
| Pharmacoat 606 | 50.4 | 27.55 |
| Klucel EF | 16.8 | 9.18 |
| Sodium Metabisulfite | 1.2 | 0.66 |
| Glycerol 99.7% | 4.8 | 2.62 |
| Transcutol HP | 8.4 | 4.59 |
| Total | 100.0 | — |

Composition 3A-2

| | | |
|---|---|---|
| Epinephrine base | 13.7 | 10.06 |
| Pharmacoat 606 | 43.5 | 31.93 |
| Klucel EF | 21.7 | 15.96 |
| Sodium Metabisulfite | 0.8 | 0.60 |
| PEG 600 | 14.0 | 10.28 |
| Transcutol HP | 6.3 | 4.59 |
| Total | 100.0 | — |

3) The Lamination of the Film Layers

The lamination of the mucoadhesive layer and the backing layer was performed using binding solution. Several alternative for lamination layers are shown below:

### Binding Solutions for Lamination:

a) Pure Ethanol 96%, or pure Ethylacetate

b) Solution of Kollidon VA 64, and PEG 600 in Ethylacetate,

| Component | Percentage (%) |
|---|---|
| Kollidon VA 64 | 3 |
| PEG 600 | 15 |
| Ethyl acetate | 82 |
| Total | 100 |

c) Solution of Kollidon VA 64, Glycerol in Etanol 96%.

| Component | Percentage (%) |
|---|---|
| Kollidon VA 64 | 3 |
| Glycerol 99.7% | 15 |
| Ethanol 96% | 82 |
| Total | 100 |

The binding solution is applied to the surface of the film layer preferably by spraying.

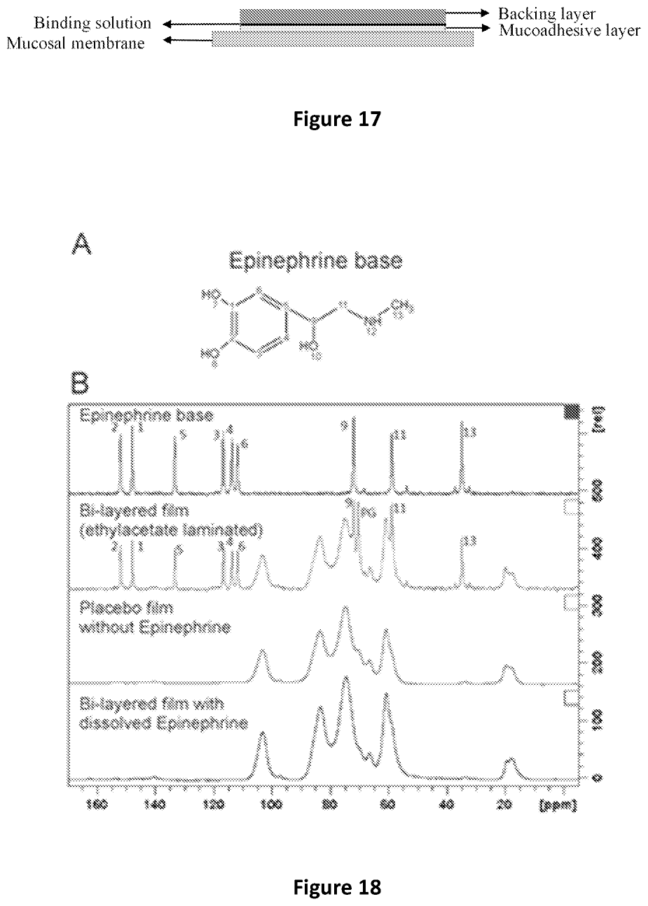

The design of the bi-layered mucoadhesive orodispersible film including a thin binding layer formed using the binding solution is presented in FIG. 17.
4) Effect of the Solvent Used on Crystallinity of Epinephrine in the Mucoadhesive Layer

NMR analysis was used to confirm crystalline state of Epinephrine base following lamination of the films.

### The Compositions Tested:

### A) Mucoadhesive Layer:

### Composition 2C-2

| Material | % of dry composition | mg/film unit |
|---|---|---|
| Epinephrine base | 18.4 | 10.06 |
| Pharmacoat 606 | 50.4 | 27.55 |
| Klucel EF | 16.8 | 9.18 |
| Sodium Metabisulfite | 1.2 | 0.66 |
| Glycerol 99.7% | 4.8 | 2.62 |
| Transcutol HP | 8.4 | 4.59 |
| Total | 100.0 | — |

### B) Binding Solution: Ethanol 96%

C) Backing Layer with Citric Acid:

| Material | % of dry composition | mg/film unit |
|---|---|---|
| Citric acid | 14.7 | 10.06 |
| Pharmacoat 606 | 46.8 | 31.93 |
| Klucel EF | 23.4 | 15.96 |
| PEG 600 | 15.1 | 10.28 |
| Total | 100.0 | — |

### D) Controls for the NMR Analysis:

As a placebo film, the same composition of mucoadhesive film layer was used in the experiment, but without Epinephrine base.

As a film formulation with dissolved Epinephrine base (solid dispersion system), the same composition of mucoadhesive film layer was used, but with addition of citric acid (10 mg/film unit) into the solution of polymers used for manufacturing of the film using solvent casting. FIG. 18 shows NMR analysis of the crystalline state of Epinephrine base in the mucoadhesive film formulations.

### E) Conclusion:

The NMR analysis of FIG. 18 confirms that the crystalline state of Epinephrine base particles was maintained in the mucoadhesive layer after the lamination step, when laminated with a citric acid-containing backing layer, and when binding solution based on 96% ethanol was used. Typical NMR signals of crystalline Epinephrine (marked by numbers 1-13) were observed using a pure Epinephrine base in the first spectrum (from top) of FIG. 18B. Typical NMR signals were also observed in the film formulation—in the second spectrum of FIG. 18 B. In both controls, a single mucoadhesive film without Epinephrine base, and single mucoadhesive film with dissolved Epinephrine, no typical signal was observed (3rd and 4th spectrum in FIG. 18 B).
5. The Dissolution of Epinephrine Base in the Film Following the Application to Oral Mucosa on the Piglet Tongue Ex Vivo

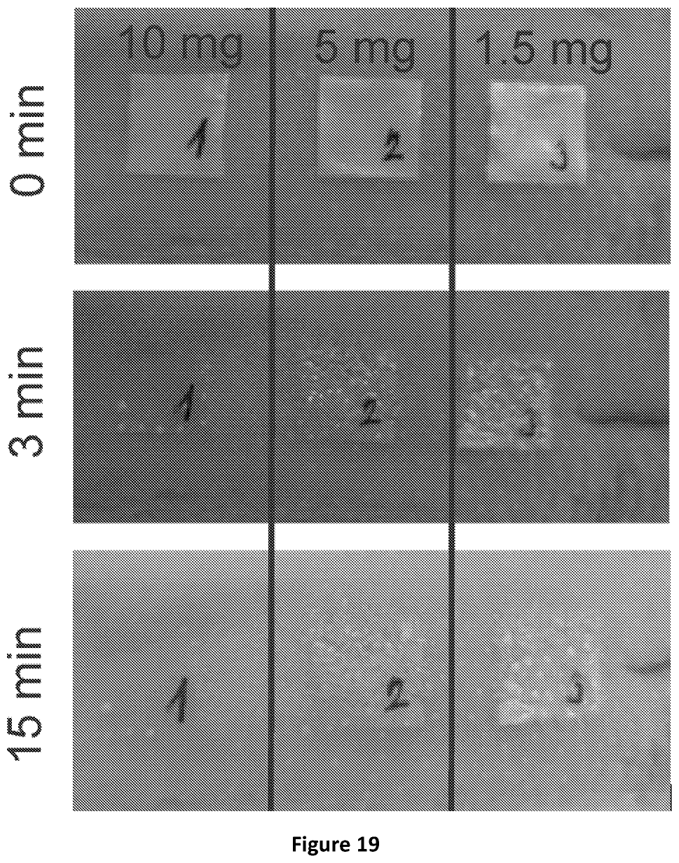

Ex vivo application of the film on the oral mucosa was performed to determine the effect of citric acid in the backing layer on the dissolution rate of the Epinephrine base in the mucoadhesive layer. The bi-layered film was applied onto porcine tongue as a model of the oral mucosa. The film was moistened by spraying a small amount of water at intervals of 2 minutes to mimic the environment in the oral cavity. The dissolution of Epinephrine base particles within the film was observable by the eye as the color of the film changed from white to transparent. The following tables describe the composition of films tested, and Fig. 19 shows the dissolution rate of Epinepbrine base particles.
A) The Composition of the Mucoadhesive Film Layer Containing Epinephrine Base Particles:

TABLE X
The mucoadhesive layer was prepared by solvent casting method
(polymer swelling/dissolution conditions: 3 hrs at 25°
C.; solvent evaporation: 4 hrs at 37° C.) using ethanol and
alkaline water mixture (80/20% v/v) 0.331 ml/cm²:

| Material | % of dry composition | mg/film unit |
|---|---|---|
| Epinephrine base | 13.7 | 10.06 |
| Pharmacoat 606 | 43.5 | 31.93 |
| Klucel EF | 21.7 | 15.96 |
| Sodium Metabisulfite | 0.8 | 0.60 |
| PEG 600 | 14.0 | 10.28 |
| Transcutol HP | 6.3 | 4.59 |
| Total | 100.0 | — |

B) The Composition of the Backing Film Layer Containing pH Modifier Citric Acid (10 mg/Film, 5 mg/Film, 1.5 mg/Film):

TABLE XI
The backing layer was prepared by solvent casting method
(polymer swelling/dissolution conditions: 3 hrs at 25°
C.; solvent evaporation: 4 hrs at 37° C.) using ethanol
and water mixture (80/20% v/v) 0.331 ml/cm²:

| | | |
|---|---|---|
| Material | % of dry composition | mg/film unit |

1. Backing layer with citric acid (10 mg/film)

| | | |
|---|---|---|
| Citric acid | 14.7 | 10.06 |
| Pharmacoat 606 | 46.8 | 31.93 |
| Klucel EF | 23.4 | 15.96 |
| PEG 600 | 15.1 | 10.28 |
| Total | 100.0 | — |

2. Backing layer with citric acid (5 mg/film)

| | | |
|---|---|---|
| Citric acid | 8.0 | 5.03 |
| Pharmacoat 606 | 50.5 | 31.93 |
| Klucel EF | 25.2 | 15.96 |
| PEG 600 | 16.3 | 10.28 |
| Total | 100.0 | — |

3. Backing layer with citric acid (1.5 mg/film)

| | | |
|---|---|---|
| Citric acid | 2.6 | 1.53 |
| Pharmacoat 606 | 53.5 | 31.93 |
| Klucel EF | 26.7 | 15.96 |
| PEG 600 | 17.2 | 10.28 |
| Total | 100.0 | — |

FIG. 19 shows dissolution of Epinephrine in the mucoadhesive layer following the application of mucoadhesive film with the citric acid-containing backing layer onto oral mucosa ex vivo (porcine tongue as a model oral mucosa). The the amount of citric acid in the backing layer of the trested film is noted at the top (film 1, 10 mg; film 2, 5 mg and film 3, 1.5 mg).
C) Conclusion:

Crystalline state of Epinephrine base in the film can be easily observed (white color) as shown in the FIG. 19, time 0 min. When dissolved, the film becomes transparent. The presence of citric acid (pH modifier) in the backing layer helps in dissolving Epinephrine base particles in the mucoadhesive layer of the film formulation. The amount of citric acid affects the dissolution rate. 10 mg of citric acid per film was shown to be sufficient to completely dissolve 10 mg Epinephrine base particles in 3 minutes after administration of the film ex vivo, whereas 5 mg of citric acid completely dissolved Epinephrine in 15 minutes, and finally, 1.5 mg of citric acid was insufficient to completely dissolve Epinpehrine base in 15 minutes.

### Example 8

### Preparation of Film Compositions with Lidocaine Hydrochloride and/or Prilocaine Hydrochloride, Epinephrine Base and Absorption Enhancers

Various film formulations were prepared to show compatibility of Lidocaine HCl with Prilocaine HCl, Epinephrine base, and selected absorption enhancers and film-forming excipients in the formulation. Two-layered design of the film was prepared: each of prepared two-layered films consisted of 1) a backing layer and 2) a mucoadhesive active pharmaceutical ingredient (API)-containing layer.

Non-Adhesive Backing Layers were Prepared by:
1) spraying a 2.5% ethanolic solution of Ethylcellulose (Ethocel™, Colorcon Limited, UK) with 2% Dibutyl phthalate (DBP, Merck KgaA, Germany) directly onto the surface of a mucoadhesive layer as an example of the non-soluble layer;
2) using the solvent casting method using ethanol/water solution or water with the combination of Hypromellose 2910 (Pharmacoat® 606, ShinEtsu, Japan) and Hydroxypropyl Cellulose (Klucel™ EF PHARM, Ashland, USA) in a 2:1 or 3:1 (w/w) ratio. Glycerol 99.5% was used at 1.8-6.7% (w/w) as plasticizer as an example of a soluble backing layer. The backing layer was then laminated with the mucoadhesive layer using a binding solution as exemplified in example 7.3) above.
Mucoadhesive API-containing layers were prepared by solvent casting method using ethanolic water or water solution with the combination of Hypromellose 2910 (Pharmacoat® 606, ShinEtsu, Japan) and Hydroxypropyl Cellulose (Klucel™ EF PHARM, Ashland, USA) in a 2:1 or 3:1 (w/w) ratio. Glycerol 99.5% was used at 1.8-6.7% (w/w) as plasticizer. Transcutol® HP, Sodium deoxycholate, Eugenol, Labrasol, Disodium ethylenediaminetetraacetic acid salt or combinations thereof were added as absorption enhancers. The combination mixture was then treated by sonication to remove air bubbles and required volumes were poured out into plastic dishes. Organic solvent/water mixture or water used as solvent were removed by evaporation at temperature 37° C./60° C. leading to the formation of the desired mucoadhesive API-containing layers.
The following table describes examples of the composition layers containing Lidocaine Hydrochloride Prilocaine Hydrochloride, Epinephrine base and absorption enhancers:

TABLE XIIa
Mucoadhesive layer prepared by solvent casting method (polymer
swelling/dissolution conditions: 3 hrs at 25° C.; solvent
evaporation: 24 hrs at 37° C.) using water 0.331 ml/cm²

| Material | % of dry composition | mg/cm² film |
|---|---|---|
| Lidocaine Hydrochloride | 31.77 | 8.28 |
| Prilocaine Hydrochloride | 31.77 | 8.28 |
| Pharmacoat ® 606 | 18.55 | 4.84 |
| Klucel ™ EF | 9.28 | 2.42 |
| Epinephrine base | 0.95 | 0.25 |
| Sodium metabisulfite | 0.06 | 0.01 |
| Transcutol HP | 5.08 | 1.33 |
| Glycerol | 2.54 | 0.66 |
| Total | 100.00 | 26.07 |

TABLE XIIb
Mucoadhesive layer prepared by solvent casting method (polymer
swelling/dissolution conditions: 3 hrs at 25° C.; solvent
evaporation: 24 hrs at 37° C.) using water 0.331 ml/cm²

| Material | % of dry composition | mg/cm² film |
|---|---|---|
| Lidocaine Hydrochloride | 30.12 | 8.28 |
| Prilocaine Hydrochloride | 30.12 | 8.28 |
| Pharmacoat ® 606 | 17.59 | 4.84 |
| Klucel ™ EF | 8.80 | 2.42 |
| Epinephrine base | 0.90 | 0.25 |
| Sodium metabisulfite | 0.05 | 0.01 |
| Transcutol HP | 10.00 | 2.75 |
| Glycerol | 2.41 | 0.66 |
| Total | 100.00 | 27.49 |

TABLE XIIc
Mucoadhesive layer prepared by solvent casting method (polymer
swelling/dissolution conditions: 3 hrs at 25° C.; solvent
evaporation: 24 hrs at 37° C.) using water 0.331 ml/cm²

| Material | % of dry composition | mg/cm² film |
|---|---|---|
| Lidocaine Hydrochloride | 32.98 | 8.28 |
| Prilocaine Hydrochloride | 32.98 | 8.28 |
| Pharmacoat ® 606 | 19.26 | 4.84 |
| Klucel ™ EF | 9.63 | 2.42 |
| Epinephrine base | 0.99 | 0.25 |
| Sodium metabisulfite | 0.06 | 0.01 |
| Deoxycholate sodium | 1.45 | 0.37 |
| Glycerol | 2.64 | 0.66 |
| Total | 100.00 | 25.11 |

TABLE XIId
Mucoadhesive layer prepared by solvent casting method (polymer
swelling/dissolution conditions: 3 hrs at 25° C.; evaporation:
24 hrs at 37° C.) using water 0.331 ml/cm²

| Material | % of dry composition | mg/cm² film |
|---|---|---|
| Lidocaine Hydrochloride | 38.20 | 14.21 |
| Prilocaine Hydrochloride | 38.20 | 14.21 |
| Pharmacoat ® 606 | 13.00 | 4.84 |
| Klucel ™ EF | 6.50 | 2.42 |
| Epinephrine base | 0.67 | 0.25 |
| Sodium metabisulfite | 0.04 | 0.01 |
| Deoxycholate sodium | 1.60 | 0.60 |
| Glycerol | 1.78 | 0.66 |
| Total | 100.00 | 37.20 |

TABLE XIIe
Mucoadhesive layer prepared by solvent casting method (polymer
swelling/dissolution conditions: 3 hrs at 25° C.; evaporation:
24 hrs at 37° C.) using water 0.331 ml/cm²

| Material | % of dry composition | mg/cm² film |
|---|---|---|
| Lidocaine Hydrochloride | 37.67 | 14.21 |
| Prilocaine Hydrochloride | 37.67 | 14.21 |
| Pharmacoat ® 606 | 12.82 | 4.84 |
| Klucel ™ EF | 6.41 | 2.42 |
| Epinephrine base | 0.66 | 0.25 |
| Sodium metabisulfite | 0.04 | 0.01 |
| Deoxycholate sodium | 2.99 | 1.13 |
| Glycerol | 1.76 | 0.66 |
| Total | 100.00 | 37.73 |

TABLE XIIf
Mucoadhesive layer prepared by solvent casting method
(polymer swelling/dissolution conditions: 3 hrs at
25° C.; solvent evaporation: 2 hrs at 60° C.) using
ethanol and alkaline water (80/20% v/v) 0.331 ml/cm²:

| Material | % of dry composition | mg/cm² film |
|---|---|---|
| Lidocaine Hydrochloride | 36.00 | 5.00 |
| Pharmacoat ® 606 | 34.80 | 4.84 |
| Klucel ™ EF | 17.40 | 2.42 |
| Epinephrine base | 1.79 | 0.25 |
| Sodium metabisulfite | 0.24 | 0.03 |
| Transcutol ® HP | 5.01 | 0.7 |
| Glycerol | 4.77 | 0.76 |
| Total | 100.00 | 14.00 |

TABLE XIIg
Mucoadhesive layer prepared by solvent casting method
(polymer swelling/dissolution conditions: 3 hrs at
25° C.; solvent evaporation: 2 hrs at 60° C.) using
ethanol and alkaline water (80/20% v/v) 0.331 ml/cm²:

| Material | % of dry composition | mg/cm² film |
|---|---|---|
| Lidocaine Hydrochloride | 33.74 | 5.00 |
| Pharmacoat ® 606 | 32.63 | 4.84 |
| Klucel ™ EF | 16.31 | 2.42 |
| Epinephrine base | 1.68 | 0.25 |
| Sodium metabisulfite | 0.22 | 0.03 |
| Transcutol ® HP | 10.28 | 1.52 |
| Glycerol | 5.14 | 0.76 |
| Total | 100.00 | 14.46 |

TABLE XIIh
Mucoadhesive layer prepared by solvent casting method
(polymer swelling/dissolution conditions: 3 hrs at
25° C.; solvent evaporation: 2 hrs at 60° C.) using
ethanol and alkaline water (80/20% v/v) 0.331 ml/cm²:

| Material | % of dry composition | mg/cm² film |
|---|---|---|
| Lidocaine Hydrochloride | 36.52 | 5.00 |
| Pharmacoat ® 606 | 35.31 | 4.84 |
| Klucel ™ EF | 17.65 | 2.42 |
| Epinephrine base | 1.81 | 0.25 |
| Sodium metabisulfite | 0.24 | 0.03 |
| Sodium deoxycholate | 3.14 | 0.43 |
| Glycerol | 5.32 | 0.73 |
| Total | 100.00 | 13.70 |

TABLE XIIi
Mucoadhesive layer prepared by solvent casting method
(polymer swelling/dissolution conditions: 3 hrs at
25° C.; solvent evaporation: 2 hrs at 60° C.) using
ethanol and alkaline water (80/20% v/v) 0.331 ml/cm²:

| Material | % of dry composition | mg/cm² film |
|---|---|---|
| Lidocaine Hydrochloride | 36.52 | 5.00 |
| Pharmacoat ® 606 | 35.31 | 4.84 |
| Klucel ™ EF | 17.65 | 2.42 |
| Epinephrine base | 1.81 | 0.25 |
| Sodium metabisulfite | 0.24 | 0.03 |
| Labrasol | 3.14 | 0.43 |
| Glycerol | 5.32 | 0.73 |
| Total | 100.00 | 13.70 |

TABLE XIIj
Mucoadhesive layer prepared by solvent casting method
(polymer swelling/dissolution conditions: 3 hrs at
25° C.; solvent evaporation: 2 hrs at 60° C.) using
ethanol and alkaline water (80/20% v/v) 0.331 ml/cm²:

| Material | % of dry composition | mg/cm² film |
|---|---|---|
| Lidocaine Hydrochloride | 35.40 | 5.00 |
| Pharmacoat ® 606 | 34.23 | 4.84 |
| Klucel ™ EF | 17.12 | 2.42 |
| Epinephrine base | 1.76 | 0.25 |
| Sodium metabisulfite | 0.23 | 0.03 |
| Eugenol | 6.10 | 0.86 |
| Glycerol | 5.16 | 0.73 |
| Total | 100.00 | 14.13 |

TABLE XIIk
Mucoadhesive layer prepared by solvent casting method
(polymer swelling/dissolution conditions: 3 hrs at
25° C.; solvent evaporation: 2 hrs at 60° C.) using
ethanol and alkaline water (80/20% v/v) 0.331 ml/cm²:

| Material | % of dry composition | mg/cm² film |
|---|---|---|
| Lidocaine Hydrochloride | 24.18 | 5.00 |
| Prilocaine Hydrochloride | 24.18 | 5.00 |
| Pharmacoat ® 606 | 23.38 | 4.84 |
| Klucel ™ EF | 11.69 | 2.42 |
| Epinephrine base | 1.20 | 0.25 |
| Sodium metabisulfite | 0.16 | 0.03 |
| Transcutol HP | 10.09 | 2.09 |
| Glycerol | 5.12 | 1.06 |
| Total | 100.00 | 20.69 |

TABLE XIIl
Mucoadhesive layer prepared by solvent casting method
(polymer swelling/dissolution conditions: 3 hrs at
25° C.; solvent evaporation: 2 hrs at 60° C.) using
ethanol and alkaline water (80/20% v/v) 0.331 ml/cm²:

| Material | % of dry composition | mg/cm² film |
|---|---|---|
| Lidocaine Hydrochloride | 31.77 | 8.28 |
| Prilocaine Hydrochloride | 31.77 | 8.28 |
| Pharmacoat ® 606 | 18.55 | 4.84 |
| Klucel ™ EF | 9.28 | 2.42 |
| Epinephrine base | 0.95 | 0.25 |
| Sodium metabisulfite | 0.06 | 0.01 |
| Transcutol HP | 5.08 | 1.33 |
| Glycerol | 2.54 | 0.66 |
| Total | 100.00 | 26.07 |

TABLE XIIm
Mucoadhesive layer prepared by solvent casting method
(polymer swelling/dissolution conditions: 3 hrs at
25° C.; solvent evaporation: 2 hrs at 60° C.) using
ethanol and alkaline water (80/20% v/v) 0.331 ml/cm²:

| Material | % of dry composition | mg/cm² film |
|---|---|---|
| Lidocaine Hydrochloride | 30.12 | 8.28 |
| Prilocaine Hydrochloride | 30.12 | 8.28 |
| Pharmacoat ® 606 | 17.59 | 4.84 |
| Klucel ™ EF | 8.80 | 2.42 |
| Epinephrine base | 0.90 | 0.25 |
| Sodium metabisulfite | 0.05 | 0.01 |
| Transcutol HP | 10.00 | 2.75 |
| Glycerol | 2.41 | 0.66 |
| Total | 100.00 | 27.49 |

TABLE XIIn
Mucoadhesive layer prepared by solvent casting method
(polymer swelling/dissolution conditions: 3 hrs at
25° C.; solvent evaporation: 2 hrs at 60° C.) using
ethanol and alkaline water (80/20% v/v) 0.331 ml/cm²:

| Material | % of dry composition | mg/cm² film |
|---|---|---|
| Lidocaine Hydrochloride | 32.98 | 8.28 |
| Prilocaine Hydrochloride | 32.98 | 8.28 |
| Pharmacoat ® 606 | 19.26 | 4.84 |
| Klucel ™ EF | 9.63 | 2.42 |
| Epinephrine base | 0.99 | 0.25 |
| Sodium metabisulfite | 0.06 | 0.01 |
| Deoxycholate sodium | 1.45 | 0.37 |
| Glycerol | 2.64 | 0.66 |
| Total | 100.00 | 25.11 |

TABLE XIIo
Mucoadhesive layer prepared by solvent casting method
(polymer swelling/dissolution conditions: 3 hrs at
25° C.; evaporation: 2 hrs at 60° C.) using ethanol
and alkaline water (80/20% v/v) 0.331 ml/cm²:

| Material | % of dry composition | mg/cm² film |
|---|---|---|
| Lidocaine Hydrochloride | 38.20 | 14.21 |
| Prilocaine Hydrochloride | 38.20 | 14.21 |
| Pharmacoat ® 606 | 13.00 | 4.84 |
| Klucel ™ EF | 6.50 | 2.42 |
| Epinephrine base | 0.67 | 0.25 |
| Sodium metabisulfite | 0.04 | 0.01 |
| Deoxycholate sodium | 1.60 | 0.60 |
| Glycerol | 1.78 | 0.66 |
| Total | 100.00 | 37.20 |

TABLE XIIp
Mucoadhesive layer prepared by solvent casting method
(polymer swelling/dissolution conditions: 3 hrs at
25° C.; evaporation: 2 hrs at 60° C.) using
ethanol and alkaline water (80/20% v/v) 0.331 ml/cm²

| Material | % of dry composition | mg/cm² film |
|---|---|---|
| Lidocaine Hydrochloride | 37.67 | 14.21 |
| Prilocaine Hydrochloride | 37.67 | 14.21 |
| Pharmacoat ® 606 | 12.82 | 4.84 |
| Klucel ™ EF | 6.41 | 2.42 |
| Epinephrine base | 0.66 | 0.25 |
| Sodium metabisulfite | 0.04 | 0.01 |
| Deoxycholate sodium | 2.99 | 1.13 |
| Glycerol | 1.76 | 0.66 |
| Total | 100.00 | 37.73 |

### Example 9

### Pharmacokinetic Study of Lidocaine Hydrochloride Containing Film of the Invention

The pharmacokinetic profile of Lidocaine Hydrochloride formulated in mucoadhesive film was determined using a pharmacokinetic model in piglets (Sus scrofa domesticus). Blood plasma concentrations of Lidocaine Hydrochloride were measured following administration of the film formulations to the gum in the area of front teeth (mandibula). The absorption of lidocaine into blood circulation confirms the local absorption into the deeper layers of the tissue. The mucoadhesive film was formulated as a bi-layered film. 1 cm×2 cm size of the film layer containing Lidocaine Hydrochloride was used in the experiment. The composition of the film layer containing Lidocaine Hydrochloride, a mucoadhesive film was applied (46 mg of Lidocaine Hydrochloride with/without 0.5 mg Epinephrine base with/without Transcutol as a absorption enhancer). The thickness of the mucoadhesive film was 160 μm.

The following formulations of Lidocaine Hydrochloride in mucoadhesive films were tested:
Mucoadhesive layer was formulated by solvent casting method (polymer swelling/dissolution conditions: 3 hrs at 25° C.; solvent evaporation: 24 hrs at 37° C.) using water 0.331 ml/cm².

### Composition of Film Containing Lidocaine HCl

| Material | % of dry composition | mg/cm² film |
|---|---|---|
| Lidocaine Hydrochloride | 77.85 | 28.41 |
| Pharmacoat ® 606 | 13.26 | 4.84 |
| Klucel ™ EF | 6.63 | 2.42 |
| Epinephrine base | 0.00 | 0.00 |
| Sodium Metabisulfite | 0.00 | 0.00 |
| Transcutol HP | 0.00 | 0.00 |
| Glycerol | 2.27 | 0.83 |
| Total | 100.00 | 36.50 |

### Composition of Film Containing Lidocaine HCl+Epinephrine Base

| Material | % of dry composition | mg/cm² film |
|---|---|---|
| Lidocaine Hydrochloride | 77.25 | 28.41 |
| Pharmacoat ® 606 | 13.15 | 4.84 |
| Klucel ™ EF | 6.58 | 2.42 |
| Epinephrine base | 0.68 | 0.25 |
| Sodium Metabisulfite | 0.09 | 0.03 |
| Transcutol HP | 0.00 | 0.00 |
| Glycerol | 2.25 | 0.83 |
| Total | 100.00 | 36.78 |

### Composition of Film Containing Lidocaine HCl+Epinephrine Base+Transcutol HP

| Material | % of dry composition | mg/cm² film |
|---|---|---|
| Lidocaine Hydrochloride | 75.22 | 28.41 |
| Pharmacoat ® 606 | 12.81 | 4.84 |
| Klucel ™ EF | 6.40 | 2.42 |
| Epinephrine base | 0.66 | 0.25 |
| Sodium Metabisulfite | 0.09 | 0.03 |
| Transcutol HP | 2.63 | 0.99 |
| Glycerol | 2.19 | 0.83 |
| Total | 100.00 | 37.77 |

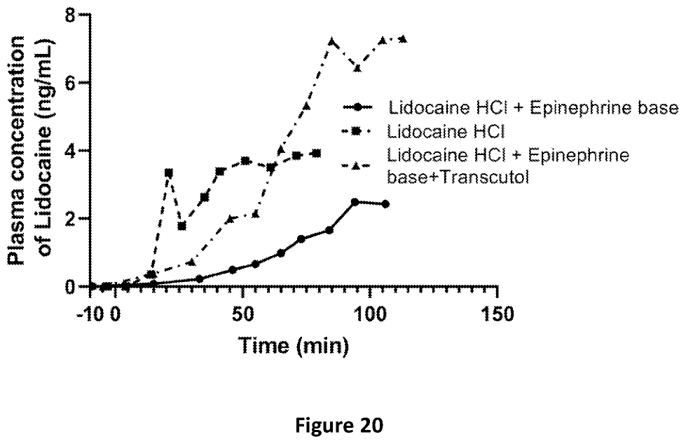

Pharmacokinetic profile of Lidocaine following administration of the prepared films is shown in FIG. 20.

### Conclusion

Effective absorption of Lidocaine through the oral mucosa of the gum was shown from the tested mucoadhesive film using a porcine animal model. Moreover, the effect of both, Transcutol as a absorption enhancer, and Epinephrine as a peripheral vasoconstrictor was clearly shown. From the data in FIG. 20 it can be concluded that:

a) Epinephrine base decreases the systemic absorption of Lidocaine from the site of absorption (gum), and

b) absorption enhancer, Transcutol®, significantly promotes local absorption leading to increase in plasma concentration of Lidocaine.

When absorption enhancer and peripheral vasoconstrictor are combined, the effect on the plasma concentration of lidocaine can be observed as shown in the graph. Therefore, the combination of local anesthetic (e.g. Lidocaine) with absorption enhancer (e.g. Transcutol), and/or peripheral vasoconstrictor (e.g. Epinephrine) can be advantageous in terms of achieving high local concentration of local anesthetic with reduced systemic absorption.

1. An oromucosal film composition comprising:
a therapeutically effective amount of epinephrine solid particles having a particle size in the range of from about 0.01 um to about 100 um; and
a pharmaceutically acceptable polymer
wherein the epinephrine solid particles are dispersed in and/or disposed on a polymeric layer.

2. The composition of claim 1 wherein the polymer is selected from the group consisting of hypromellose, hydroxypropyl cellulose, hydroxyethyl cellulose, carboxymethyl cellulose, carboxymethyl cellulose sodium, carbomer, polycarbophil, povidone, polyvinyl alcohol, polyethylene oxide, polyethylene glycol, sodium alginate, calcium alginate, xanthan gum, pectin, hyaluronic acid, sodium hyaluronate, tragacanth, guar gum, acacia gum, arabic gum, lectin, starch, gelatin, pullulan, carrageenan, chitosan, amino methacrylate copolymers, poloxamer, collagen, poly-amino acids, and mixtures thereof.

3. The composition of claim 1 wherein the epinephrine particle size is in the range of from about 10 um to about 80 um.

4. The composition of claim 1 wherein the composition further comprises one or more pharmaceutically acceptable excipients selected from the group consisting of pH modifiers, absorption enhancers, disintegrants, plasticizers, saliva stimulating agent, taste masking agents, flavoring agents, sweeting agent, coloring agent, antioxidants, stabilizers, anti-foaming and/or defoaming components and mixtures thereof.

5. The composition of claim 4 wherein the pH modifiers are selected from the group consisting of citric acid, acetic acid, succinic acid, tartaric acid, maleic acid, malic acid, ascorbic acid, acetylsalicylic acid, adipic acid, fumaric acid, glutaric acid, glutamic acid itaconic acid, aspartic acid, lactic acid, and salts and mixtures thereof.

6. The composition of claim 4 wherein the absorption enhancers are selected from the group consisting of chelators, non-ionic surfactants, cationic surfactants, anionic surfactants, bile salts and other steroidal detergents, fatty acids, fatty acids salts and esters, sucrose fatty acid esters, non-surfactants, phospholipids, complexing agents, cyclodextrins, alkyl glycosides, polyethylene glycol alkyl ethers, self-emulsifying agents, and mixtures thereof.

7. The composition of claim 4 wherein the absorption enhancers are selected from the group consisting of limonene, menthol, pinene, clove oil, eugenol, caprylocaproyl polyoxyl-8-glycerides, propylene glycol monocaprylate, deoxycholate sodium, taurocholate sodium, glycocholate sodium, diethylene glycol monoethyl ether, dodecylmaltoside, tetradecyl maltoside, and mixtures thereof.

8. The composition of claim 1 wherein the composition is a bi-layered film where a first layer comprises epinephrine solid particles dispersed in and/or disposed on a mucoadhesive polymeric layer, and a second layer which acts as a backing layer.

9. The composition of claim 1 wherein the composition is a tri-layered film comprising:
a layer comprising the epinephrine solid particles dispersed in and/or disposed on a polymeric layer;
a mucoadhesive layer; and
a backing layer,
wherein either the mucoadhesive layer or the polymeric layer comprising epinephrine particles is the middle layer.

10. The composition of claim 8 further comprising one or more local anesthetics in the polymeric layer.

11. The composition of claim 10 wherein the local anesthetic is selected from the group consisting of lidocaine, articaine, bupivacaine, prilocaine, mepivacaine and mixtures thereof.

12. A method for preparation of the composition of claim 1 wherein the method comprises solvent casting, hot melt extrusion, electrospinning, 3-dimensional or flexographic printing, spraying, combination of solvent casting and spraying, electrospraying, solution blow spinning, electroblowing, centrifugal spinning, or a combination of electrospinning and electrospraying.

13. The method of claim 12 wherein the method involves using a solvent/dispersant selected from the group consisting of lower alkyl alcohols, acetone, methyl acetate, ethyl acetate, isopropyl acetate, acetonitrile, heptane, tetrahydrofuran, water, alkaline water, aqueous buffer, and mixtures thereof.

14. A method for preparation of the composition of claim 1 wherein the method comprises:
providing a suspension of epinephrine solid particles having a particle size in the range of from about 0.01 um to about 100 um in a solvent/dispersant;
simultaneously or separately providing a suspension or solution of the polymer in a solvent/dispersant;
combining the epinephrine solid particle suspension and the polymer suspension/solution if prepared separately; and
removing the solvent/dispersant to obtain the polymeric layer.

15. The method of claim 14 wherein the solvent is selected from the group consisting of lower alkyl alcohols, acetone, methyl acetate, ethyl acetate, isopropyl acetate, acetonitrile, heptane, tetrahydrofuran, water, alkaline water, aqueous buffer, and mixtures thereof.

16. A method of treating anaphylactic shock, cardiac arrest, asthma, bronchial asthma, bronchitis, emphysema, respiratory infections, or allergic reactions in a subject in need thereof comprising administering to the subject a composition according to claim 1.

17. The method of claim 16 wherein the composition is administered sublingually or buccally.

18. A method of providing local anesthesia or treating tooth pain or mouth ulcers in a subject in need thereof comprising administering to the subject a composition according to claim 10.

19. An oromucosal film composition comprising:
a therapeutically effective amount of epinephrine solid particles having a particle size in the range of from about 0.01 um to about 100 um; and
a pharmaceutically acceptable polymer,
wherein the epinephrine solid particles are dispersed in and/or disposed on a polymeric layer, and wherein the composition excludes an anti-crystallization agent elected from the group consisting of sugar alcohols and di-alcohols.

20. An oromucosal film composition consisting essentially of:
a therapeutically effective amount of epinephrine solid particles having a particle size in the range of from about 0.01 um to about 100 um;
one or more absorption enhancers;
one or more pH modifiers; and
a pharmaceutically acceptable polymer
wherein the epinephrine solid particles are dispersed in and/or disposed on a polymeric layer.
